(19) United States
(12) Patent Application Publication
(10) Pub. No.: US 2026/0108502 A1
(43) Pub. Date: Apr. 23, 2026

(54) ORAL DELIVERY FORMULATIONS USING SELF EMULSIFYING GRANULES AND LIPID BASED GRANULES

(71) Applicant: BOARD OF REGENTS, THE UNIVERSITY OF TEXAS SYSTEM, Austin, TX (US)

(72) Inventors: Mohammed MANIRUZZAMAN, Oxford, MS (US); Bhupendra RAJ GIRI, Austin, TX (US); Vineet R. KULKARNI, Austin, TX (US)

(73) Assignee: BOARD OF REGENTS, THE UNIVERSITY OF TEXAS SYSTEM, Austin, TX (US)

(21) Appl. No.: 19/364,498

(22) Filed: Oct. 21, 2025

Related U.S. Application Data
(60) Provisional application No. 63/710,474, filed on Oct. 22, 2024.

(51) Int. Cl. A61K 31/427; A61K 9/16; A61P 35/00

(52) U.S. Cl. CPC A61K 31/427; A61K 9/1617; A61K 9/1641; A61K 9/1694; A61P 35/00

(57) ABSTRACT

The present disclosure provides compositions and methods of preparing compositions comprising using lipid containing pharmaceutical compositions that are prepared using an additive manufacturing technique such as a sintering or melting process. The pharmaceutical compositions may form self emulsifying drug delivery systems. The pharmaceutical compositions may form lipid coated drug particles that are formed from the melting of the lipid through the additive manufacturing process.

This application claims the benefit of U.S. Provisional Patent Application No. 63/710,474, filed Oct. 22, 2024, the entirety of which is incorporated herein by reference.

### BACKGROUND OF THE INVENTION

### 1. Field

The present disclosure relates generally to the field of pharmaceuticals and pharmaceutical manufacture. More particularly, it concerns compositions and methods of preparing lipid-based compositions using additive manufacturing techniques such as selective laser melting (SLM) or others.

### 2. Description of Related Art

Since the early 1970s, additive manufacturing (AM) commonly referred as 3D-printing (3DP) has attracted significant interest from researchers from diverse discipline like engineering and technology, pharmaceutical science, biological science and medical devices, and so on. Over the past decade, this fascination had expanded to the pharmaceutical domain, primarily due to its potential for innovative, complex yet customized design, manufacturing and delivery, rapid prototyping, patient-specific i.e., personalized dosage forms development with tailored release profiles, multi drug-drug combination (polypill) and point-of-care manufacturing capabilities. These innovative approaches are hard to achieve with traditional mass-manufacturing methods like tableting and capsule filling. It is therefore anticipated that 3D printing is set to revolutionize a broad array of pharmaceutical applications, ranging from expediting drug development to on-demand printing of patient-specific medications in community pharmacies, hospitals in remote locations, or even outer space drugs manufacturing and delivery.

Different 3DP technologies commonly used in pharmaceutical manufacturing includes fused deposition modeling (FDM), semi-solid extrusion (SSE), stereolithography (SLA), powder bed fusion (PBF) and material jetting. Each of these 3DP techniques operates in a similar fashion i.e., to build products in a layer-by-layer printing method based on the computerized.stl file. However, these 3DP techniques uses different feedstock material for printing, for example, FDM requires filament of certain mechanical properties (diameter, flexibility and stiffness), SSE requires solvent considerations and paste of certain rheology for extrusion, SLA requires light-reactive resin with optimal rheological and thermal properties. Similarly, both the powder-based 3DP techniques like (i) powder bed fusion e.g., selective laser sintering (SLS) and selective laser melting (SLM), and (ii) material jetting e.g., drop-on demand (DOD), and binder jetting requires feedstock powder materials of certain intrinsic (thermal conductivity, rheological, and melting temperature (Tₘ)/glass transition temperature (T_{g})) and extrinsic properties for example good flowability and spread properties, smooth and spherical morphology, and narrow particle size distribution (PSD) and drug distribution. Pharmaceutical drug and excipients are readily available in powder form, which makes powder-based 3DP techniques as an attractive choice over other 3DP methods. However, many drug powder materials are generally cohesive in nature and do not possess good flow properties, have needle- or rod-like morphology with wide PSD, and different intrinsic properties than excipients which limits the use of PBF 3DP technology for pharmaceutical manufacturing. In addition, poor flowability arising from fine and cohesive powder results in poor micromeritic properties like, tapped density and bulk density, angle of repose (AOR), compressibility index (CI), which necessitates the pre-processing of feedstock materials or diluting with flow enhances or other excipients. Failure to improve micromeritic properties often results in uneven powder layering, segregation thereby, inefficient sintering of drug-carrier particles, leading to layering of printed parts “printless”, friable dosage forms with insufficient hardness, high porosity, uneven and low drug distribution in the final dosage forms. It is therefore apparent to have a suitable feedstock material for PBF 3DP method. Another ongoing challenge that must be addressed is the rapid disintegration and drug release from the tablets or printlets created using PBF-based printing techniques, such as SLS, due to their inherently porous nature and loosely structured composition. To prevent potential drug/excipient degradation induced by high thermal energy from lasers-based SLS (for example, CO₂ lasers having λ=˜10,600 nm), researchers prefer to use lower-intensity, low-cost diode lasers operating at visible/NIR wavelengths (λ=445 750 nm) for manufacturing pharmaceutical dosage forms. However, the energy density which is a function of laser power, hatch distance, scanning speed and layer thickness, is not sufficient enough for efficient sintering or fusion or bonding between drug-carrier particles, leading to friable printlets with high porosity, fast disintegration and immediate drug release. Fina et al. (2018) demonstrated that ACH 3D-printed tablets containing HPMC E5 and Kollidon® VA 64 exhibited a porous structure, resulting in faster disintegration and accelerated drug release profiles. In our previous work (Giri et al., 2022), the fabrication of an SLS 3D-printed sustained-release acetaminophen tablet (a model drug) using Kollidone SR® as a matrix former and employing the 2.3-W blue diode laser operating at visible wavelength (SINTRATEC Kit, AG, Brugg, Switzerland, λ=445 nm) was reported. However, the compositions were only able to achieve a 10% drug-loading and had to use a higher concentration (5%) of Candurin® pigment for thermal conduction and fusion of drug-polymer particles. Even at the highest energy density printing conditions (i.e., low laser speed and low hatch distance), an initial burst release of the drug (approximately 55% drug release within the first two hours of the dissolution experiment) was observed. Moreover, the total porosity in the tablet observed through micro-CT images (including both open and closed pores) was relatively high, which also contributed to the initial higher drug release. This highlights the need for the development of alternative manufacturing technologies or the investigation of other carriers suitable for fabricating PBF-based 3D printed oral dosage forms with adequate strength, minimal or reduced total porosity in the final printed tablets, and the overall objective of preventing initial burst drug release while achieving an extended drug release profile.

SLM, also known as Direct Metal Laser Sintering (DMLS), is another PBF technique similar to SLS, that uses a laser to fully melt the powder particles, creating a homogeneous, fully dense material with higher hardness. Smooth surface finish, and low porosity. Where SLS sinters or fuses the particles by heating the drug-carrier powder material below the Tₘ or T_{g} or preferably between its T_{g} and Tₘ of binary mixture, without fully melting but creating a bond or neck between particles, but in SLM, the laser heats the powder to its melting point, allowing the particles to completely fuse together and form a solid structure (FIG. 1). This partial melting process in SLS results in parts with a porous microstructure, which can be advantageous for specific applications, such as creating scaffolds for tissue engineering (Williams et al., 2015). The resulting parts may have slightly lower mechanical strength and less isotropic properties compared to those produced by SLM (Yap et al., 2015). SLS parts typically have a rougher surface finish, which may require additional post-processing steps to achieve the desired surface quality (Gibson et al., 2015). Since the powder particles are fully melted in SLM, the resulting parts have higher mechanical strength and more isotropic properties compared to those produced by SLS (Yap et al., 2015). SLM parts generally have a smoother surface finish, although some post-processing may still be required to achieve the desired surface quality (Gibson et al., 2015).

Therefore, there remains a need to develop new methods of preparing suitable compositions with lipids that can then be used in additive manufacturing techniques to create better quality including dense and low porosity 3D-printed products.

### SUMMARY OF THE INVENTION

In some aspects, the present disclosure provides methods of preparing compositions that contain one or more active pharmaceutical ingredients with one or more lipid components using additive manufacturing techniques.

In some aspects, the present disclosure provides methods of preparing a pharmaceutical composition comprising:
(A) obtaining a precursor composition comprising:
   (1) one or more active pharmaceutical ingredient;
   (2) one or more lipid components; and
   (3) one or more energy absorbing material;
(B) extruding the precursor composition in an extruder to obtain a feedstock composition; and
(C) subjecting the feedstock composition to a sintering process in an additive manufacturing to obtain the pharmaceutical composition.

In some embodiments, the methods further comprise subjecting the precursor solution to a sieve before extrusion. In some embodiments, the particle size of the sieved powder contains a multitude of size that have a diameter of at least 100 μm. In some embodiments, the particle size have a diameter of at least 200 μm such as of about 250 μm.

In some embodiments, the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives. In some embodiments, the active pharmaceutical ingredient is a BCS class I drug. In some embodiments, the active pharmaceutical ingredient is a BCS class II drug. In some embodiments, the active pharmaceutical ingredient is a BCS class III drug. In some embodiments, the active pharmaceutical ingredient is a BCS class IV drug. In some embodiments, the active pharmaceutical ingredient is a nonsteroidal anti-inflammatory agent.

In some embodiments, the pharmaceutical composition comprises one lipid. In some embodiments, the pharmaceutical composition comprises two or more lipids. In some embodiments, the lipid is a glyceride lipid. In some embodiments, the glyceride lipid comprises two fatty acids. In other embodiments, the glyceride lipid comprises three fatty acids. In some embodiments, the glyceride lipid comprises a fatty acid with an aliphatic carbon tail. In some embodiments, the glyceride lipid comprises a fatty acid with an aliphatic carbon tail comprising from about 6 to about 30 carbon atoms. In some embodiments, the aliphatic carbon tail comprises from about 12 carbon atoms to about 24 carbon atoms such as from about 18 carbon atoms to about 24 carbon atoms. In some embodiments, the glyceride lipid is glyceryl dibehenate.

In some embodiments, the electromagnetic energy-absorbing excipient is a material that leads to improved energy absorption. In some embodiments, the electromagnetic energy-absorbing excipient is a material with a lambda max (Amax) equal to the wavelength of the laser. In some embodiments, the lambda max is from about 50 nm to about 15,000 nm. In some embodiments, the lambda max is from about 200 nm to about 11,000 nm such as from about 200 nm to about 1,000 nm. In some embodiments, the energy absorbing excipient is an inorganic material. In some embodiments, the energy absorbing excipient is an aluminum material. In some embodiments, the aluminum material is an aluminum inorganic salt. In some embodiments, the aluminum inorganic salt is bentonite, potassium aluminum silicate, aluminum, aluminum sulfates, sodium aluminum phosphate acidic, sodium aluminum silicate, calcium aluminum silicate, starch aluminum octenyl succinate, or potassium aluminum silicate with a coating of titanium dioxide and/or iron oxide. In some embodiments, the aluminum inorganic salt is potassium aluminum silicate with a coating of titanium dioxide and/or iron oxide. In some embodiments, the inorganic material is iron oxide, titanium oxide, or silicates. In some embodiments, the energy absorbing excipient is an organic material. In some embodiments, the organic material is a dye such as carmine, a phthalocyanine, or a diazo compound.

In some embodiments, the precursor composition comprises an amount of the active pharmaceutical ingredient from about 1% to about 98%. In some embodiments, the amount of the active pharmaceutical ingredient from about 30% to about 95%. In some embodiments, the amount of the active pharmaceutical ingredient from about 50% to about 90%. In some embodiments, the precursor composition comprises an amount of the one or more lipids from about 1% to about 70%. In some embodiments, the amount of the one or more lipids from about 5% to about 60%. In some embodiments, the amount of the one or more lipids from about 10% to about 50%. In some embodiments, the precursor composition comprises an amount of the energy absorbing excipient from about 0.1% to about 10% w/w. In some embodiments, the amount of the energy absorbing excipient is from about 0.5% to about 5%. In some embodiments, the amount of the energy absorbing excipient is from about 1% to about 2.5%. In some embodiments, the amount of the energy absorbing excipient is about 1.5%.

In some embodiments, the extruder is a hot melt extruder. In some embodiments, the hot melt extruder is a twin-screw hot melt extruder. In some embodiments, the extruding is carried out with a screw speed is from about 5 rpm to about 200 rpm. In some embodiments, the screw speed is from about 10 rpm to about 150 rpm. In some embodiments, the screw speed is from about 20 rpm to about 125 rpm. In some embodiments, the extruding is carried out with a feed rate from about 1 g/min to about 20 g/min. In some embodiments, the feed rate is from about 2.5 g/min to about 15 g/min. In some embodiments, the feed rate is from about 5 g/min to about 10 g/min. In some embodiments, the feed rate is about 8 g/min.

In some embodiments, the precursor composition is passed through a sieve before extrusion to form a sieved powder. In some embodiments, the particle size of the sieved powder contains a multitude of particles with a diameter from about 10 μm to about 1 mm. In some embodiments, the particle size is from about 100 μm to about 500 μm. In some embodiments, the particle size is from about 200 μm to about 300 μm such as about 250 μm. In some embodiments, the feedstock composition is passed through a sieve after extrusion. In some embodiments, the feedstock composition is passed through two or more sieves after extrusion. In some embodiments, the feedstock composition is passed through two sieves after extrusion. In some embodiments, the sieve comprises a pore diameter from about 10 μm to about 500 μm. In some embodiments, the pore diameter is from about 50 μm to about 250 μm. In some embodiments, the pore diameter is from about 100 μm to about 150 μm such as about 125 μm. In other embodiments, the sieve comprises a pore diameter from about 5 μm to about 250 μm. In some embodiments, the pore diameter is from about 25 μm to about 100 μm. In some embodiments, the pore diameter is from about 50 μm to about 75 μm such as about 65 μm.

In some embodiments, the melting process is a selective laser melting process. In some embodiments, the melting or sintering process comprises using a laser to heat the feedstock composition. In some embodiments, the melting or sintering process comprises using a laser to melt the feedstock composition. In some embodiments, the laser comprises a power from about 100 mW to about 20 W. In some embodiments, the power is from about 500 mW to about 10 W. In some embodiments, the power is from about 1 W to about 5 W such as about 2.3 W. In some embodiments, the laser has a wavelength from about 250 nm to about 1100 nm. In some embodiments, the wavelength is from about 300 nm to about 700 nm. In some embodiments, the wavelength is from about 400 to about 500 nm such as about 455 nm.

In some embodiments, the sintering process comprises using a laser with a hatch spacing from about 5 μm to about 150 μm. In some embodiments, the hatch spacing is from about 10 μm to about 100 μm. In some embodiments, the hatch spacing is from about 25 μm to about 75 μm. In some embodiments, the hatch spacing is 50 μm. In some embodiments, the sintering process comprises using a laser with a hatching offset from about 5 μm to about 500 μm. In some embodiments, the hatching offset is from about 25 μm to about 250 μm. In some embodiments, the hatching offset is from about 100 μm to about 150 μm such as 120 μm.

In some embodiments, the sintering process comprises using a laser with a number of perimeters from about 0 to about 10. In some embodiments, the number of perimeters is from about 1 to about 5. In some embodiments, the number of perimeters is from about 1 to about 3. In some embodiments, the number of perimeters is 1. In some embodiments, the sintering process comprises using a laser with a perimeter offset from about 10 μm to about 750 μm. In some embodiments, the perimeter offset is from about 50 μm to about 500 μm. In some embodiments, the perimeter offset is from about 100 μm to about 300 μm such as 200 μm. In some embodiments, the sintering process comprises applying a layer of feedstock composition with a layer thickness from about 10 μm to about 750 μm. In some embodiments, the layer thickness is from about 50 μm to about 500 μm. In some embodiments, the layer thickness is from about 100 μm to about 300 μm such as 150 μm.

In some embodiments, the sintering process comprises passing a laser over the feedstock composition with a laser speed from about 5 mm/s to about 150 mm/s. In some embodiments, the laser speed is from about 10 mm/s to about 100 mm/s. In some embodiments, the laser speed is from about 25 mm/s to about 75 mm/s. In some embodiments, the laser speed is 50 mm/s.

In some embodiments, the melting or sintering process comprises a chamber with a chamber temperature that is below the melting point or glass transition temperature of the feedstock composition. In some embodiments, the chamber temperature is less than 150° C. In some embodiments, the chamber temperature is less than 100° C. In some embodiments, the chamber temperature is less than 75° C. In some embodiments, the chamber temperature is less than 50° C. In some embodiments, the melting process comprises a chamber with a layer of the feedstock composition applied to a print surface. In some embodiments, the print surface has a surface temperature. In some embodiments, the print surface temperature is near or below the melting point or glass transition temperature of the feedstock composition. In some embodiments, the print surface temperature is less than 200° C. In some embodiments, the print surface temperature is less than 100° C. In some embodiments, the print surface temperature is less than 75° C. In some embodiments, the print surface temperature is less than 50° C. In some embodiments, the melting process comprises applying a layer of the feedstock composition to a surface in a chamber. In some embodiments, the melting process comprises collecting the feedstock composition that has not been exposed to a laser. In some embodiments, the melting process comprises applying a second layer of the feedstock composition after a laser has been passed over the first layer of the feedstock composition. In some embodiments, melting process comprises repeating the applying of another layer of the feedstock composition after the laser has been passed over the first layer of the feedstock composition multiple times. In some embodiments, the repeating is done from 5 times to 1,000 times. In some embodiments, the repeating is done from 10 times to 100 times.

In another aspect, the present disclosure provides compositions comprising at least two layers of a composition comprising:
(1) an active pharmaceutical ingredient;
(2) one or more lipid components; and
(3) an energy absorbing material.

In yet another aspect, the present disclosure provides methods of preparing a pharmaceutical composition comprising:
(A) admixing a lipid and a BCS Class II or BCS Class IV drug at an elevated temperature to form a precursor solution; and
(B) extruding the pharmaceutical composition to obtain one or more granules of the pharmaceutical composition.

In some embodiments, the elevated temperature is sufficient to melt the lipid. In some embodiments, the admixing further comprises a surfactant. In some embodiments, the surfactant is a polymer or comprises a polymer component. In some embodiments, the surfactant is a polymer. In some embodiments, the surfactant is a polyethylene glycol or polypropylene glycol. In some embodiments, the surfactant is a polyethylene glycol. In some embodiments, the surfactant is a polymer having a molecular weight from about 100 to about 10,000. In some embodiments, the molecular weight is from about 750 to about 7,500. In some embodiments, the molecular weight is from about 1000 to about 6000. In some embodiments, the surfactant is a surfactant that comprises a polymer component. In some embodiments, the surfactant is a sorbitol conjugated with one or more polyethylene glycol units or one or more polypropylene glycol and one or more hydrophobic groups. In some embodiments, the surfactant comprises one or more polyethylene glycol units. In some embodiments, the one or more hydrophobic groups are one or more lipid groups. In some embodiments, the surfactant is a tween compound. In some embodiments, the surfactant is a sorbitol comprising one or more hydrophobic groups. In some embodiments, the one or more hydrophobic group is a lipid.

In some embodiments, the surfactant and lipid are present in the precursor solution are present in a ratio from about 1:20 to about 5:1. In some embodiments, the ratio is about 1:15 to about 2:1. In some embodiments, the ratio is about 1:10 to about 1:1. In some embodiments, the lipid is a mixture of one or more monoglycerides, one or more diglycerides, or one or more triglycerides. In some embodiments, the lipid is a mixture of one or more monoglycerides, one or more diglycerides, and one or more triglycerides. In some embodiments, the mixture further comprises one or more polyethylene glycol or polypropylene glycol polymers. In some embodiments, the lipid comprises one or more fatty acid groups covalently linked to a polyethylene glycol polymer. In some embodiments, the lipid comprises a fatty acid group with a carbon atom count from 8 carbon atoms to 24 carbon atoms. In some embodiments, the fatty acid group has a carbon atom count from about 12 carbon atoms to about 18 carbon atoms. In some embodiments, the fatty acid group has a carbon atom count from about 18 carbon atoms to about 24 carbon atoms. In some embodiments, the lipid has a polyethylene glycol polymer or a polypropylene polymer with a molecular weight from about 500 to about 10,000. In some embodiments, the molecular weight is from about 1,000 to about 7,500. In some embodiments, the molecular weight is from about 1,000 to about 2,000. In some embodiments, the precursor composition comprises from about 10% to about 90% by weight of the lipid. In some embodiments, the precursor composition comprises from about 30% to about 80% by weight of the lipid. In some embodiments, the precursor composition comprises from about 50% to about 70% by weight of the lipid.

In some embodiments, the precursor composition comprises a BCS Class II drug. In some embodiments, the precursor composition comprises a BCS Class IV drug. In some embodiments, the BCS Class II or BCS Class IV drug is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives. In some embodiments, the precursor composition comprises from about 10% to about 90% by weight of the drug. In some embodiments, the precursor composition comprises from about 20% to about 70% by weight of the drug. In some embodiments, the precursor composition comprises from about 30% to about 50% by weight of the drug.

In some embodiments, the elevated temperature is greater than 30° C. In some embodiments, the elevated temperature is greater than 50° C. In some embodiments, the elevated temperature is from about 30° C. to about 150° C. In some embodiments, the elevated temperature is from about 50° C. to about 100° C. In some embodiments, the methods comprise mixing at a speed of greater than 25 rpm. In some embodiments, the speed is greater than 50 rpm. In some embodiments, the mixing is performed at a speed from about 10 rpm to about 250 rpm. In some embodiments, the speed is from about 25 rpm to about 200 rpm. In some embodiments, the speed is from about 50 rpm to about 150 rpm. In some embodiments, the extrusion comprises using a screw extruder. In some embodiments, the screw extruder is a twin-screw extruder. In some embodiments, the extrusion occurs with two or more distinct temperature zones. In some embodiments, the extrusion occurs with two, three, or four distinct temperature zones. In some embodiments, the extrusion occurs in three distinct temperature zones. In some embodiments, a first distinct temperature zone has a first zone temperature from about 20° C. to about 80° C. In some embodiments, the first zone temperature is from about 30° C. to about 60° C. such as about 40° C. In some embodiments, a second distinct temperature zone has a second zone temperature from about 0° C. to about 150° C. In some embodiments, the second zone temperature is from about 20° C. to about 130° C. such as about 70° C. In some embodiments, a third distinct temperature zone has a second zone temperature from about 0° C. to about 50° C. In some embodiments, the third zone temperature is from about 5° C. to about 30° C. such as about 10° C.

In some embodiments, the extrusion comprises a processing speed from about 2.5 rpm to about 100 rpm. In some embodiments, the processing speed is from about 5 rpm to about 50 rpm. In some embodiments, the processing speed is from about 30 rpm. In some embodiments, the extrusion comprises a feeding rate from about 1 g/mm to about 10 g/mm. In some embodiments, the feeding rate is from about 2 g/mm to about 5 g/mm such as about 3 g/mm.

In some embodiments, the methods further comprise converting the pharmaceutical compositions into a powder. In some embodiments, the methods further comprise subjecting the pharmaceutical composition to an additive manufacturing process. In some embodiments, the additive manufacturing process is a powder-based additive manufacturing process. In some embodiments, the additive manufacturing process is direct powder extrusion 3D printing. In some embodiments, the additive manufacturing process comprises a printing speed from about 1 mm/s to about 20 mm/s. In some embodiments, the printing speed is from about 2.5 mm/s to about 10 mm/s. In some embodiments, the printing speed is about 5 mm/s. In some embodiments, the additive manufacturing process comprises a print pressure from about 10 kPa to about 1 MPa. In some embodiments, the print pressure is from about 50 kPa to about 500 kPa such as about 150 kPa. In some embodiments, the additive manufacturing process comprises applying a powder through a needle. In some embodiments, the needle is a 20-gauge needle. In some embodiments, the additive manufacturing process comprises producing a final drug product. In some embodiments, the final drug product is an oral dosage form. In some embodiments, the oral dosage form is a tablet.

In another aspect, the present disclosure provides compositions prepared according to the methods described herein.

In still another aspect, the present disclosure provides methods of treating a disease or disorder comprising administering to the patient a therapeutically effective amount of a composition described herein.

In yet another aspect, the present disclosure provides methods of preventing a disease or disorder comprising administering to the patient a therapeutically effective amount of a composition described herein.

In still another aspect, the present disclosure provides methods of treating a disease or disorder comprising administering to the patient a therapeutically effective amount of a composition according to the methods described herein.

In still yet another aspect, the present disclosure provides methods of preventing a disease or disorder comprising administering to the patient a therapeutically effective amount of a composition prepared according to the methods described herein. In some embodiments, the composition comprises a drug which treats or prevents the corresponding disease or disorder.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1 shows a schematic illustration of the overview of the work.

FIG. 2 shows the saturation solubility of ritonavir (RTV) in lipid matrices.

FIG. 3 shows the phase diagrams prepared for the four selected screening combinations of (G48/16-PEG3350), (G48/16+G44/14 (1:1)-PEG3350), (G48/16-PEG6000), and (G48/16+G44/14 (1:1)-PEG6000).

FIG. 4 shows the particle size and PDI analysis of samples collected at 80%, 85%, 90%, 95% and 99% water content during phase diagram analysis for G48/16-PEG3350 mixture.

FIGS. 5A-5D shows the solid-state analysis (A) thermogravimetric analysis (TGA), (B) differential scanning calorimetry, (C) X-ray diffraction analysis, and (D) polarized light microscopy, for pure drug RTV, G48/16, PEG3350 and final formulated granules using optimized extrusion process post DOE studies.

FIG. 6 shows the rheology analysis of lipid (G48/16), physical mixture and formulation (granules).

FIG. 7 shows the hot-melt extrusion screw design and temperature profile set up for granule development and DoE study space.

FIG. 8 shows the contour plots for effect of processing conditions on response variables.

FIG. 9 shows the digital images of granules processed under different conditions as per DOE output.

FIG. 10 shows the NMR analysis of pure drug (RTV), lipid (G48/16) and formulated granules.

FIGS. 11A & 11B show the release performance comparison for (A) pure drug, physical mixture, and formulation in DI water and (B) pH shift study for comparing formulation performance against pure drug.

FIGS. 12A-12D show the SEM morphology of (A,C) 30% drug loaded granules and (B,D) 40% drug loaded granules.

FIG. 13 shows the release performance comparison for 30% and 40% drug loaded granules.

FIG. 14 shows the release performance comparison of SNEDDS prepared using extruded granules and SNEDDS prepared using conventional method.

FIG. 15 shows the release performance comparison of tablets prepared using direct powder extrusion with physical mixture as raw feed against granules prepared using extruded granules as raw feed.

FIG. 16 shows the cell viability of ritonavir-loaded SMEDDS and blank SMEDDS at different concentrations of lipids across Caco-2 cells for 4 h incubated at 37° C. Data are expressed as mean±SD (n=5). * p<0.001 vs. control, ** p<0.001 vs. control, and *** p<0.001 vs. control.

FIG. 17 shows the in vitro permeability profiles of ritonavir released from SMEDDS across Caco-2 cell monolayers. All experiments were performed from the apical to the basolateral direction in HBSS-HEPES buffer (pH 7.4) at 37° C. Data are expressed as mean±SD (n=3).

FIGS. 18A-18B show the effect of lipid (binder) level on granulation properties (% mass fraction and flow properties) of different formulations at fixed HME conditions (screw speed 50 rpm and P3 temperature profile).

FIGS. 19A and 19B show the dino-lite microscopy images of the neat formulation components, physical mixture (PM), and prepared HME granules of different formulations (FA1 to FA4 compostions).

FIGS. 20A and 20B shows the scanning electron microscopy images of neat formulation components, physical mixture (PM), and prepared HME granules of different formulations (FA1 to FA4 compositions).

FIG. 21 shows dino-lite microscopy, polarized light microscopy (PLM), and scanning electron microscopy (SEM) images of the neat formulation components, and prepared HME granules of different formulations (FA1 to FA4 compositions).

FIGS. 22A and 22B shows the dino-lite microscopy images of the prepared 3D-printed tablets and the average weight of tablets from different formulations (FA1 to FA4 compositions).

FIG. 23 shows the drug release profiles from the 3D-printed tablets of different compositions.

FIG. 24 shows the micro-CT images of the whole and half-sliced 3D-printed tablet (FA2 formulation top and bottom view) post 24 hours of dissolution studies.

FIGS. 25A and 25B show the PXRD graphs for the neat formulation components, physical mixture (PM) and 3D-printed tablet of different compositions (FA1 to FA4).

FIG. 26 shows the DSC thermograms of the neat formulation components and 3D-printed tablet of different compositions (FA1 to FA4).

FIG. 27 shows the FT-IR spectra of the neat formulation components and 3D-printed tablet of different compositions (FA1 to FA4).

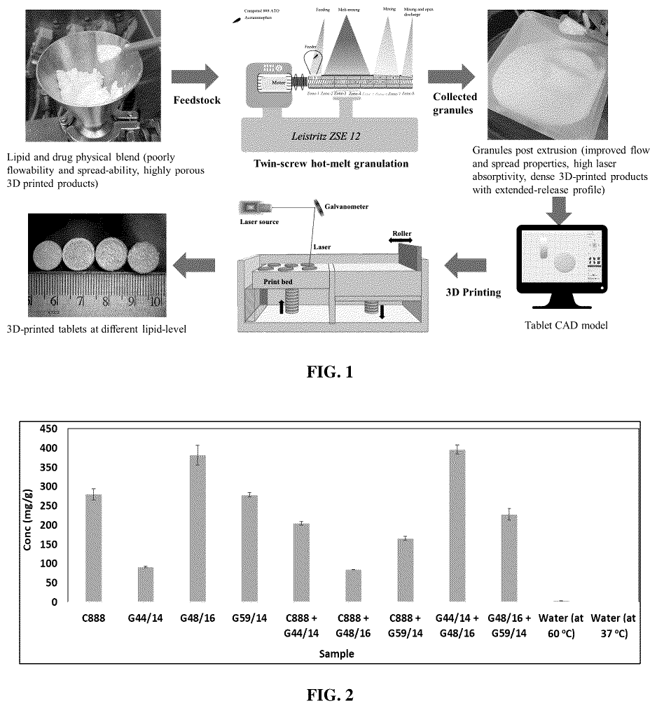

### DESCRIPTION OF ILLUSTRATIVE EMBODIMENTS

The present disclosure relates to the use of additive manufacturing techniques such as a single-step extrusion process, for formulating lipid-based solid granules that could be then delivered by filling into capsules or as 3D printed tablets using a direct powder extrusion system. These granules would then self-emulsify post-delivery to improve the apparent solubility and permeability of the active pharmaceutical ingredient. Combining the capabilities of hot-melt extrusion to form drug loaded homogeneous granules as a continuous process along with personalizing the medications using the layer-by-layer printing capabilities of powder-based 3D printing techniques or other attitie manufacturing techniques such as direct powder extrusion would improve the drug delivery prospects for such candidates.

Gelucires® are a group of lipid excipients composed of mono-, di-, and triglycerides and mono- and di-fatty acid esters of polyethylene glycol (PEG) (Panigrahi et al., 2018). These are available with a range of properties depending on their HLB and melting point range of 33 to 65° C., with a wide variety of applications in oral and topical formulations (Nyavanandi et al., 2023; Wang et al., 2023). These excipients have a distinctive composition of lipids, surfactants and cosurfactants, because of which they serve different functions such as water-soluble and water-insoluble surfactants and matrix forming agents. All of these Gelucires are categorized as Generally Recognized as Safe (GRAS) excipients. Some examples of these nonionic gelucires are Gelucire 44/14, Gelucire 48/16 and Gelucire 59/14 (Panigrahi et al., 2018; Kale and Patravale, 2008). The chemical composition of these are listed in Table-1. Gelucire containing only PEG esters are generally used in the preparation of fast/immediate/rapid release formulations (Upadhyay et al., 2013). Gelucire containing only glycerides, or a mixture of glycerides and PEG esters are used in the preparation of sustained release formulations (Panigrahi et al., 2018; Upadhyay et al., 2013). Owing to their extreme hydrophobicity, are considered as appropriate carriers for designing sustained release drug delivery systems. Based on HLB values, gelucires can be classified into hydrophilic and hydrophobic grades. Gelucire exhibiting HLB value less than 6 can be called hydrophobic; 6 to 9 are water dispersible and above 9 can be recognized as hydrophilic grade (Panigrahi et al., 2018; Wang et al., 2023; Kale and Patravale, 2008; Upadhyay et al., 2013).

Ritonavir (RTV) is a protease inhibitor used in a combination drug therapy to treat HIV (human immunodeficiency virus) infection in adults and children. It is a BCS Class IV with a strongest acidic and basic pKa of 13.68 and 2.84, respectively (Chandwani and Shuter, 2008; Law et al., 2001). This poor solubility and permeability makes oral delivery of this drug a challenge and hence was used as a model drug for the study.

Hot-melt extrusion (HME) is an effective process known for its adaptability, durability, and wide-ranging utility within the pharmaceutical sector, especially for its capability to function without the requirement of organic solvents, thereby eliminating the need for post-dispersion solvent removal (Maniruzzaman et al., 2012). This attribute offers several advantages, including a reduction in processing stages, minimized environmental footprint, and recognition as an eco-friendly technology (Sarabu et al., 2021; Maniruzzaman et al., 2012). Compared to the conventionally used processes for melt-mixing for preparing lipid based systems, HME surpasses those in achieving superior mixing due to its continuous and controlled process (Sarabu et al., 2019; Farinha et al., 2020; Chauhan et al., 2020). HME provides precise temperature and shear control, ensuring homogeneous dispersion of ingredients, especially in pharmaceutical and polymer industries (Maniruzzaman et al., 2012).

Direct powder extrusion (DPE) 3D printing involves the extrusion of material through the nozzle of a printer in the form of a semi-solid paste or gel based on the raw feed in the nozzle (Rosch et al., 2023). The physical blend of the final formulation can be directly fed to the printer to print the final formulation in the desired shape in a layer-by-layer manner (Pistone et al., 2023). Use of physical blends often leads to the segregation of individual components under pressure, leading to non-uniform drug distribution (Henry et al., 2021; Wang et al., 2023). This can lead to overdosing or underdosing which can cause harmful side-effects, especially in the case of potent low dose drugs. Use of granules having uniformly dispersed drug in a homogenous matrix would help overcome the above challenge and improve the final printed formulation (Bhujbal et al., 2021; McDonagh et al., 2022).

These and other advantages are discussed in more detail below.

### I. Pharmaceutical Compositions

In some aspects, the present disclosure provides methods of preparing a pharmaceutical compositions containing a lipid component, an active pharmaceutical ingredient, and one or more energy absorbing material.

The pharmaceutical compositions described herein comprise an active pharmaceutical ingredient. The pharmaceutical compositions described herein contain an active pharmaceutical ingredient in an amount between about 0.1% to about 95% w/w, between about 0.25% to about 90% w/w, between about 0.5% to about 50% w/w, or between about 1% to about 40% w/w of the total composition. In some embodiments, the amount of the active pharmaceutical ingredient is from about 0.1%, 0.2%, 0.25%, 0.3%, 0.4%, 0.5%, 0.6%, 0.7%, 0.75%, 0.8%, 0.9%, 1%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33% 34%, 35%, 36%, 37%, 38%, 39%, 40%, 42%, 44%, 45%, 46%, 48%, 50%, 52%, 54%, 55%, 56%, 58%, 60%, 65%, 70%, 75%, 80%, to about 90% w/w or any range derivable therein. In some embodiments, the pharmaceutical composition is substantially, essentially, or entirely free of any other compounds.

### A. Active Pharmaceutical Ingredients

In some embodiments, the active pharmaceutical ingredient is classified using the Biopharmaceutical Classification System (BCS), originally developed by G. Amidon, which separates pharmaceuticals for oral administration into four classes depending on their aqueous solubility and their permeability through the intestinal cell layer. According to the BCS, drug substances are classified as follows: Class I-High Permeability, High Solubility; Class II-High Permeability, Low Solubility; Class III-Low Permeability, High Solubility; and Class IV-Low Permeability, Low Solubility.

While the pharmaceutical compositions and methods described herein can be applied to any BCS class of drugs, BCS class II and IV are of interest for the pharmaceutical compositions described herein. Additionally, other active pharmaceutical ingredients that are of specific consideration are those are those that are high melting point drugs such as a drug that has a melting point of greater than 60° C. Alternatively, the active pharmaceutical ingredients used herein may have a melting point from about 35° C. to about 1,000° C., from about 50° C. to about 750° C., or from about 60° C. to about 200° C. In particular, the melting point may be greater than 25° C. 35° C., 50° C., 60° C., 80° C., 100° C., 125° C., 150° C., 175° C., 200° C., or 250° C. In particular, the composition may also comprise drugs that are laser sensitive. Such active pharmaceutical ingredients may be formulated with other compounds such as dyes that increase the stability of these active pharmaceutical ingredients.

Suitable active pharmaceutical ingredients may be any poorly water-soluble, biologically active pharmaceutical ingredients or a salt, isomer, ester, ether or other derivative thereof, which include, but are not limited to, anticancer agents, antifungal agents, psychiatric agents such as analgesics, consciousness level-altering agents such as anesthetic agents or hypnotics, nonsteroidal antiinflammatory agents (NSAIDS), anthelminthics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, antiinflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, hormones, immunosuppressants, keratolytics, lipid regulating agents, leukotriene inhibitors, macrolides, muscle relaxants, neuroleptics, nutritional agents, opioid analgesics, protease inhibitors, or sedatives.

In some aspects, the method may be mostly used with active pharmaceutical ingredients which undergo degradation at an elevated temperature or pressure/shear. The active pharmaceutical ingredients that may be used include those which decompose at a temperature above about 50° C. In some embodiments, the active pharmaceutical ingredients decompose above a temperature of 80° C. In some embodiments, the active pharmaceutical ingredients decompose above a temperature of 100° C. In some embodiments, the active pharmaceutical ingredients decompose above a temperature of 150° C. The active pharmaceutical ingredients that may be used include therein which decompose at a temperature of greater than about 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., or 150° C.

Alternatively, active pharmaceutical ingredients may be one that is sensitive to shear. These active pharmaceutical ingredients are compounds for which the chemical and/or physical properties may change due to friction resulting from the manufacturing process itself, including chemical degradation of a drug or the loss of molecular weight of a polymer as non-limiting examples. The degree of loss of the chemical or physical properties of a compound due to shear is often seen as a function of the degree of mixing (e.g., blade RPM, rotation speed) and the properties of the polymer carrier (e.g. rheological properties).

### B. Polymer Formulation

In some aspects, the present disclosure relates to compositions that may be used as resins to prepare devices and dosage forms. The polyalkylene units may result from the polymerization of a C2-C4 alkylene monomer. In particular, the polymerization may be of an ethylene, propylene, or butylene monomer. In particular, the polymerization is of an ethylene monomer resulting in polyethylene. The polymers may comprise a weight from about 100 to about 50,000, from about 500 to about 25,000, or from about 1,000 to about 10,000. The weight of the polymer may be from about 100, 250, 500, 750, 1,000, 2,500, 5,000, 7,500, 10,000, 25,000, 50,000, 75,000, to about 100,000, or any range derivable therein.

The polymers that may be used in the present applications may comprise from about 0.5% by weight to about 80% by weight of the vinyl ester in the polymer. The amount of the vinyl ester in the polymer may be from about 0.5%, 1%, 2%, 2.5%, 3%, 4%, 5%, 6%, 7%, 7.5%, 8%, 9%, 10%, 11%, 12%, 12.5%, 13%, 14%, 15%, 16%, 17%, 17.5%, 18%, 19%, 20%, 21%, 22%, 22.5%, 23%, 24%, 25%, 26%, 27%, 27.5%, 28%, 29%, 30%, 32.5%, 35%, 40%, 45%, 50%, 55%, 60%, 65%, 70%, 75%, to about 80% by weight, or any range derivable therein.

### C. Lipids

In some aspects, the present disclosure provides pharmaceutical compositions containing one or more lipid, oil, or solvent. As used herein, a lipid or oil is a hydrophobic molecule which exhibits fat-like solubility and is largely insoluble in water. The lipid or oil may be a fatty acid, a triglyceride, an ester of a fatty acid, or mixtures thereof. The term lipid includes fatty acids which are a group of aliphatic saturated or unsaturated carboxylic acids. The chains are usually unbranched and have 6 to 30, preferably 8 to 22, and in particular 8 to 18, carbon atoms. Some non-limiting examples of saturated fatty acids include caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, behenic acid, lignoceric acid, cerotic acid and melissic acid. Additionally, the term includes unsaturated fatty acids may be unsaturated one or more times, in particular unsaturated once, twice, three times, four times, five times or six times. Some non-limiting examples of singly unsaturated fatty acids include palmitoleic acid, oleic acid and erucic acid, of doubly unsaturated fatty acids include sorbic acid and linoleic acid, of triply unsaturated fatty acids include linolenic acid and eleostearic acid, of quadruply unsaturated fatty acids include arachidonic acid, of quintuply unsaturated fatty acids include clupanodonic acid, and of sextuply unsaturated fatty acids include docosahexaenoic acid.

Alternatively, the terms lipid and oil may include glycerides which are esters of glycerol. Depending on the number of ester groups, the glyceride may be referred to as a mono-, di- and triglycerides. The acid residue in a monoglyceride may be at position 1 or 2 and the acid residues of di- and triglycerides may be identical or different and be distributed in every conceivable way over the three possible positions of glycerol. The acid residues are preferably the fatty acids described above. Examples of monoglycerides include glycerol monobehenate, glycerol monocaprate, glycerol monococoate, glycerol monoerucate, glycerol monoisostearate, glycerol monolanolate, glycerol monolaurate, glycerol monolinoleate, glycerol monomyristate, glycerol monooleate, glycerol monopalmitate, glycerol monoricinoleate, glycerol monostearate, of the diglycerides include glycerol dicaprylate, glycerol dilaurate, glycerol dimyristate, glycerol dioleate, glycerol dipalmitate and glycerol distearate, of the triglycerides include glycerol tricaprylate, glycerol trilaurate, glycerol trimyristate, glycerol trioctanoate, glycerol trioleate, glycerol triricinoleate and glycerol tristearate. Many common pharmaceutical oils and lipids comprises one or more glycerides and these pharmaceutical oils and lipids include Capmul, CapTex, and Labrafac. Additionally, other oils or lipids may include esters of fatty acids such as methyl palmitate, ethyl linoleate, or isopropyl palmitate.

In some aspects, the amount of each lipid=is from about 1% to about 20% w/w, from about 2% to about 10% w/w, from about 2% to about 8% w/w, or from about 2% to about 4% w/w. The amount of each lipid comprises from about 1%. 1.25%, 1.5%, 1.75%, 2%, 3%, 4%, 5%, 6%, 7%, 8%, 99%, 10%, 12%, 14%, 15%, 16%, 18%, 20%, 22%, 25%, 28%, 30%, 32%, 35%, 38%. 40%, 45%, to about 50% w/w, or any range derivable therein, of the total pharmaceutical composition. In one embodiment, the amount of the lipid is at 2% to 5% w/w of the total weight of the pharmaceutical composition.

i. Surfactants

In some aspects, the present disclosure provides pharmaceutical compositions may further comprise one or more surfactants. In some embodiments, the compositions have either one or two surfactants. As used herein, the term surfactant refers to a compound which exhibits amphiphilic character and reduces the surface tension of a solvent, particularly water. Surfactants can generally be classified into four categories: cationic, anionic, zwitterionic, or non-ionic. While it is contemplated that any of these surfactants may be used in the present compositions, non-ionic surfactant show particular promise. Cationic surfactants include, but are not limited to, amines with long alkyl chains and are protonated at a physiologically relevant pH or permanently charged quaternary ammonium salts such as cetrimonium bromide, cetylpyridinium chloride, benzalkonium chloride, benzethonium chloride, dimethyldioctadecylammonium chloride, or dioctadecyldimethylammonium bromide. Some non-limiting examples of anionic surfactants include sulfate, sulfonate, or phosphate esters such as docusate, perfluorooctanesulfonate, perfluorobutanesulfonate, alkyl-aryl ether phosphates, or alkyl ether phosphate or carboxylate esters including alipahtic carboxylates such as fatty acids and derivatives thereof. Other examples of zwitterionic surfactants including phospholipids such as phosphotidylserine, phosphotidylcholine, phosphotidylethanolamine, or sphingomyelins, sultaines such as CHAPS and cocamidopropyl hydroxysultaine, or betaine such as cocamidopropyl betaine. Finally, some non-limiting examples of nonionic surfactants include PEG alkyl ethers, polypropylene glycol ethers, glucoside alkyl ethers, PEG alkylaryl ethers such as Triton® and nonoxynol, simple alkyl esters of glycerol such as glycerol laurate, polysorbates such as Tween, Sorbitan alkyl esters such as Span, or poloxamer and other block copolymers of polyethylene glycol and polypropylene glycol. In some embodiments, the surfactants used in the present pharmaceutical compositions contain one or more polyethylene glycol or polypropylene glycol polymer such as Tween, Capryol, Labrafil, or Labrasol.

In some aspects, the amount of the surfactant is from about 1% to about 20% w/w, from about 2% to about 10% w/w, from about 2% to about 8% w/w, or from about 2% to about 4% w/w. The amount of the surfactant comprises from about 1%, 1.25%, 1.5%, 1.75%, 2%, 3%, 4%, 5%, 6%, 7%, 8%, 9%, 10%, 12%, 14%, 15%, 16%, 18%, to about 20% w/w, or any range derivable therein, of the total pharmaceutical composition. In one embodiment, the amount of the surfactant is at 2% to 5% w/w of the total weight of the pharmaceutical composition.

### D. Other Excipients

In some aspects, the present disclosure provides pharmaceutical compositions that may further comprise one or more additional excipients. The excipients (also called adjuvants) that may be used in the presently disclosed compositions and composites, while potentially having some activity in their own right, for example, antioxidants, are generally defined for this application as compounds that enhance the efficiency and/or efficacy of the active pharmaceutical ingredient. It is also possible to have more than one active agent in a given solution so that the particles formed contain more than one active agent. In particular, the compositions may further comprise one or more flowability excipients such as a silicon compound. The silicon compound may include an oxide of silicon such as silicon dioxide.

Any pharmaceutically acceptable excipient known to those of skill in the art may be used to produce the pharmaceutical compositions disclosed herein. Examples of excipients for use with the present disclosure include, lignin, gelatin methacrylate, lactose, glucose, starch, calcium carbonate, kaolin, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerine and starch, lactose, bentonite, colloidal silicic acid, talc, stearates, and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, vitamin E TPGS, polyoxyethylated glycolysed glycerides, dipalmitoyl phosphadityl choline, glycolic acid and salts, deoxycholic acid and salts, sodium fusidate, cyclodextrins, polyethylene glycols, polyglycolyzed glycerides, polyvinyl alcohols, polyacrylates, polymethacrylates, polyvinylpyrrolidones, phosphatidyl choline derivatives, cellulose derivatives, biocompatible polymers selected from poly(lactides), poly(glycolides), poly(lactide-co-glycolides), poly(lactic acid) s, poly(glycolic acid) s, poly(lactic acid-co-glycolic acid) s and blends, combinations, and copolymers thereof.

As stated, excipients and adjuvants may be used in the pharmaceutical composition to enhance the efficacy and efficiency of the active agent in the pharmaceutical composition. Additional non-limiting examples of compounds that can be included are binders, carriers, cryoprotectants, lyoprotectants, surfactants, fillers, stabilizers, polymers, protease inhibitors, antioxidants, bioavailability enhancers, and absorption enhancers. The excipients may be chosen to modify the intended function of the active ingredient by improving flow, or bioavailability, or to control or delay the release of the API. Specific nonlimiting examples include: sucrose, trehalose, Span 80, Span 20. Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor® EL, Cremophor® RH, Gelucire® 50/13, Gelucire® 53/10, Gelucire® 44/14, Labrafil®, Solutol® HS, dipalmitoyl phosphatidyl choline, glycolic acid and salts, deoxycholic acid and salts, sodium fusidate, cyclodextrins, polyethylene glycols, Labrasol®, polyvinyl alcohols, polyvinyl pyrrolidones, and tyloxapol. In particular, the composition may further comprise one or more silicon compounds such as silicon dioxide that improves the flowability of the composition.

The stabilizing carrier may also contain various functional excipients, such as: hydrophilic polymer, antioxidant, super-disintegrant, surfactant including amphiphilic molecules, wetting agent, stabilizing agent, retardant, similar functional excipient, or a combination thereof, and plasticizers including citrate esters, polyethylene glycols, PG, triacetin, diethyl phthalate, castor oil, and others known to those of ordinary skill in the art. Extruded material may also include an acidifying agent, adsorbent, alkalizing agent, buffering agent, colorant, flavorant, sweetening agent, diluent, opaquing, complexing agent, fragrance, preservative or a combination thereof.

Compositions with enhanced solubility may comprise a mixture of the active pharmaceutical ingredient and an additive that enhances the solubility of the active pharmaceutical ingredient. Examples of such additives include but are not limited to surfactants, polymer-carriers, pharmaceutical carriers, thermal binders, or other excipients. A particular example may be a mixture of the active pharmaceutical ingredient with a surfactant or surfactant, the active pharmaceutical ingredient with a polymer or polymers, or the active pharmaceutical ingredient with a combination of a surfactant and polymer carrier or surfactants and polymer-carriers. A further example is a composition where the active pharmaceutical ingredient is a derivative or analog thereof.

In some embodiments, the pharmaceutical compositions may further comprise one or more surfactants. Surfactants that can be used in the disclosed pharmaceutical compositions to enhance solubility include those known to a person of ordinary skill. Some particular non-limiting examples of such surfactants include but are not limited to sodium dodecyl sulfate, dioctyl docusate sodium, Tween 80, Span 20, Cremophor® EL or Vitamin E TPGS.

Solubility can be indicated by peak solubility, which is the highest concentration reached of a species of interest over time during a solubility experiment conducted in a specified medium at a given temperature. The enhanced solubility can be represented as the ratio of peak solubility of the agent in a pharmaceutical composition of the present disclosure compared to peak solubility of the reference standard agent under the same conditions. Preferably. an aqueous buffer with a pH in the range of from about pH 4 to pH 8, about pH 5 to pH 8, about pH 6 to pH 7, about pH 6 to pH 8, or about pH 7 to pH 8, such as, for example, pH 4.0, 4.5, 5.0, 5.5, 6.0, 6.2, 6.4, 6.6, 6.7, 6.8, 6.9, 7.0, 7.1, 7.2, 7.4, 7.6, 7.8, or 8.0, may be used for determining peak solubility. This peak solubility ratio can be about 2:1, 3:1, 4:1, 5:1, 6:1, 7:1, 8:1, 9:1, 10:1, 12:1, 15:1, 20:1, 25:1, 30:1, 35:1, 40:1, 45:1, 50:1, 55:1 or higher.

Compositions of the active pharmaceutical ingredient that enhance bioavailability may comprise a mixture of the active pharmaceutical ingredient and one or more pharmaceutically acceptable adjuvants that enhance the bioavailability of the active pharmaceutical ingredient. Examples of such adjuvants include but are not limited to enzyme inhibitors. Particular examples are such enzyme inhibitors include but are not limited to inhibitors that inhibit cytochrome P-450 enzyme and inhibitors that inhibit monoamine oxidase enzyme. Bioavailability can be indicated by the Cₘₐₓ or the AUC of the active pharmaceutical ingredient as determined during in vivo testing, where Cₘₐₓ is the highest reached blood level concentration of the active pharmaceutical ingredient over time of monitoring and AUC is the area under the plasma-time curve. Enhanced bioavailability can be represented as the ratio of Cₘₐₓ or the AUC of the active pharmaceutical ingredient in a pharmaceutical composition of the present disclosure compared to Cₘₐₓ or the AUC of the reference standard the active pharmaceutical ingredient under the same conditions. This Cₘₐₓ or AUC ratio reflecting enhanced bioavailability can be about 5:1, 6:1, 7:1, 8:1, 9:1, 10:1, 12:1, 15:1, 20:1, 25:1, 30:1, 35:1, 40:1, 45:1, 50:1, 55:1, 60:1, 65:1, 70:1, 75:1, 80:1, 85:1, 90:1, 95:1, 98:1, 99:1, 100:1 or higher.

In other aspects, the present compositions may further comprise one or more opacifying agents. Opacifying agents include such compounds as titanium oxide and alter the clarity and ability of energy to be absorbed by the compositions. Alternatively, these compositions may alter the amount of energy needed to achieve appropriate processing of the compositions. Some non-limiting examples of opacfying agents include those taught by U.S. Pat. Nos. 4,009,139, 5,571,334, and PCT Patent Application No. WO 2020/122950, the entire contents of which are hereby incorporated by reference. Some non-limiting examples of opacifying agents including Aerosil®, Cab-O Si®, or other silicon dioxides, aluminum hydroxide, alumina, aluminum silicate, arachidic acid, barium sulfate, bentonite, calamine, calcium carbonate, calcium phosphate dibasic, calcium phosphate tribasic, calcium silicate, calcium sulfate, ceric oxide, cetyl alcohol, activated charcoal, charcoal, diatomaceous earth, erucamide, ethylene glycol monosterate, Fuller's earth, guanine, hectorite, kaolin, magnesium aluminum silicate, magnesium carbonate, magnesium oxide, magnesium phosphate tribasic, magnesium silicate, magnesium trisilicate, myristic acid, palmitic acid, silica, stannic oxide, stearic acid amide, stearoyl monoethanolamine sterate, stearyl palmitate, talc, titanium dioxide, Veegum® or other granular magnesium aluminum silicates, zinc carbonate basic, zirconium oxide, or zirconium silicate. In other aspects, these excipients may be a light absorbing excipient such as a dye or fluorophore. Some non-limiting examples of dyes include xanthene, BOPIDY, coumarin, cypate, or other well known conjugated systems of multiple bonds.

In some aspects, the amount of the excipient in the pharmaceutical composition is from about 0.1% to about 20% w/w, from about 0.25% to about 10% w/w, from about 0.5% to about 7.5% w/w, or from about 0.5% to about 5% w/w. The amount of the excipient in the pharmaceutical composition comprises from about 0.1%, 0.2%, 0.25%, 0.3%, 0.4%, 0.5%, 0.6%. 0.7%, 0.75%, 0.8%, 0.9%, 1%, 1.25%, 1.5%, 1.5%, 1.75%, 2%, 2.5%, 3%, 3.5%, 4%, 4.5%, 5%, 5.5%, 6%, 6.5%, 7%, 7.5%, 8%, 9%, to about 10% w/w, or any range derivable therein, of the total pharmaceutical composition. In one embodiment, the amount of the excipient in the pharmaceutical composition is at 0.25% to 2.5% w/w of the total weight of the pharmaceutical composition.

### II. Extrusion Methods

Thus, in one aspect, the present disclosure provides pharmaceutical compositions which may be prepared using a thermal or fusion-based high energy process. Such process may include hot melt extrusion, hot melt granulation, melt mixing, spray congealing, sintering/curing, injection molding, or a thermokinetic mixing process such as the KinetiSol method. Similar thermal processing methods are described in LaFountaine et al., 2016a, Keen et al., 2013, Vynckier et al., 2014, Lang et al., 2014, Repka et al., 2007, Crowley et al., 2007, DiNunzio et al., 2010a, DiNunzio et al., 2010b, DiNunzio et al., 2010c. DiNunzio et al., 2010d, Hughey et al., 2010, Hughey et al., 2011, LaFountaine et al., 2016b, and Prasad et al., 2016, all of which are incorporated herein by reference. In some embodiments of these present disclosure, the pharmaceutical compositions may be prepared using a thermal process such as hot melt extrusion or hot melt granulation. In other embodiments, a fusion based process including thermokinetic mixing process such as those described at least in U.S. Pat. Nos. 8,486,423 and 9,339,440, the entire contents of which are herein incorporated by reference.

A non-limiting list of instruments which may be used to thermally process the pharmaceutical compositions described herein include hot melt extruders available from ThermoFisher, such as a minilab compounder, or Leistritz, such as a twin-screw extruder. Alternatively, a fusion-based high energy process instrument that does not require external heat input, including such as a thermokinetic mixer as described in U.S. Pat. Nos. 8,486,423 and 9,339,440 may be used to process the pharmaceutical composition.

In some aspects, the extruder may comprise heating the composition to a temperature from about 60° C. to about 250° C. In some embodiments, the temperature is from about 100° C. to about 200° C. The temperature that may be used is from about 60° C., 65° C., 70° C. 75° C., 80° C., 90° C., 92° C., 94° C., 96° C., 98° C., 100° C., 102° C., 104° C., 106° C., 108° C., 110° C., 112° C., 114° C., 116° C., 118° C., 120° C., 125° C. 130° C., 135° C., 140° C., 145° C., 150° C., 155° C., 160° C., 165° C., 170° C. 175° C., 180° C., 190° C., 200° C. 225° C., to about 250° C. or any range derivable therein.

In some embodiments, the extruder comprises a screw speed from about 5 rpm to about 200 rpm, from about 10 rpm to about 150 rpm, or from about 20 rpm to about 125. In some embodiments, the screw speed is 5 rpm, 10 rpm, 20 rpm, 25 rpm, 50 rpm, 60 rpm, 75 rpm, 80 rpm, 100 rpm, 110 rpm, 125 rpm, 140 rpm, 150 rpm, to about 200 rpm, or any range derivable therein. The extursion is carried out at with a feed rate 1 g/min to about 20 g/min or from about 2.5 g/min to about 15 g/min. In some embodiments, the feed rate is about 1 g/min, 2.5 g/min, 5 g/min. 7.5 g/min, 10 g/min, 12.5 g/min, 15 g/min, 17.5 g/min, 20 g/min, 22.5 g/min, to about 25 g/min, or any range derivable therein.

The extrudate may be in the form of granules of a desired mesh size or diameter, rods that can be cut and shaped into tablets, and films of a suitable thickness that shaped forms can be punched into suitable size and shape for administration. This extrudate may be used in further processing steps to yield the final pharmaceutical product or composition. The extrudate of the pharmaceutical composition may be dried, formed, milled, sieved, or any combination of these processes to obtain a final composition which may be administered to a patient. Such processes are routine and known in the art and include formulating the specific product to obtain a final pharmaceutical or nutraceutical product. Additionally, the extrudate of the pharmaceutical composition obtained may be processed using a tablet press to obtain a final table. Additionally, it may be milled and combined with one or more additional excipients to form a capsule or pressed into a table. The resultant pharmaceutical composition may also be dissolved in a solvent to obtain a syrup, a suspension, an emulsion, or a solution.

In some embodiments, the methods further comprise passing the pharmaceutical composition after extrusion through a sieve and obtaining a pharmaceutical composition that has a pore diameter from about 10 μm to about 500 μm, from about 50 μm to about 250 μm, or from about 100 μm to about 150 μm. In some embodiments, the hatching offset is 10 μm, 20 μm, 25 μm, 30 μm, 40 μm, 50 μm, 60 μm, 70 μm, 75 μm, 80 μm, 90 μm, 100 μm, 125 μm, 150 μm, 200 μm, 250 μm, 300 μm, 400 μm, 500 μm, 600 μm, 700 μm, to about 750 μm, or any range derivable therein.

### III. Additive Manufacturing Methods

Various additive manufacturing including additive systems are contemplated in the present disclosure. These additive manufacturing or additive systems represent machines used for additive manufacturing. These machines operate under various process categories and are used to refer to a category of machines rather than a particular commercial vendor variation of process methodology. Several examples of additive manufacturing process categories include: binder jetting, directed energy deposition, material extrusion, material jetting, powder bed fusion, sheet lamination, and vat photopolymerization. Vat photopolymerization refers to an additive manufacturing process in which liquid photopolymer in a vat is selectively cured by light-activated polymerization. Similarly, stereolithography is a vat photopolymerization process used to produce parts from photopolymer materials in a liquid state using one or more lasers to selectively cure to a predetermined thickness and harden the material into shape layer upon layers. In particular, stereolithography apparatus is an SLA additive machine. As a non-limiting example, SL additive machines are commercially available via various vendors, such as Envision Tec 3D, Formlabs, 3D systems corporation, etc.

The devices described herein are cured products of the resin composition for stereolithography. Specifically, the flexible AM devices of the present invention are cured by irradiating the polymer composition for stereolithography with laser/light. While the hardness, tensile strength at break, tensile elongation at break, and compression of the flexible shaped biomedical devices made using the pharmaceutical composition may be set appropriately according to the mechanical properties required for the product.

The flexible shaped biomedical devices made according to the methods described herein may be formed into any desired shapes by a stereolithography method. These flexible shaped biomedical devices may be produced by any known stereolithography methods, using the compositions described herein as raw material.

The method for producing the flexible biomedical devices can be suitably performed by using the composition described herein, in a conventionally known stereolithography method that uses a liquid resin as a raw material, such as being used in various stereolithography methods including LCD (stereolithography liquid display method: Liquid Crystal Display), DLP (stereolithography projector (surface exposure) method: Digital Light Processing), and SLA (stereolithography laser method: Stereolithography Apparatus).

In some aspects, the present disclosure provides composition that may be used to prepare biomedical devices described herein. These devices are prepared using a method including the steps of forming a first-layer cured product by supplying the compositions for stereolithography described herein onto a stereolithography platform, and irradiating the polymer composition for stereolithography with light/laser to cure the polymer composition for stereolithography: forming a second-layer cured product by supplying the composition described gereub for producing the second-layer cured product onto the first-layer cured product, and irradiating the compositions for stereolithography with laser/light to cure the polymer composition for stereolithography; and repeating the same step as the step of forming the second-layer cured product until a final layer is formed to devices (AM parts) with a three-dimensional shape. For a stereolithography method, any known 3D printer may be used and several types of 3D stereolithographic printer are commercially available.

In a stereolithography method, the thickness of a single layer upon curing the composition described herein is, for example, from about 1 micron to about 1 mm, from about 10 to about 750 microns, from about 20 to about 500 microns, or from about 25 to about 300 microns. The size of the layers may be from about 1 μm, 2.5 μm. 5 μm, 10 μm, 15 μm, 20 μm, 25 μm, 50 μm, 75 μm, 100 μm, 150 μm, 200 μm, 250 μm, 300 μm, 350 μm, 400 μm, 450 μm, 500 μm, 600 μm, 700 μm, 800 μm, 900 μm, to about 1 mm, or any range derivable therein The irradiation light is typically ultraviolet light and preferably includes light with a wavelength of 405 nm. The wavelength of light may be from about 300 nm to about 800 nm, from about 350 nm to about 600 nm, or from about 400 nm to about 500 nm. Similarly, the laser used may have a power from about 10 mW to about 1 W, from about 50 nW to about 750 mW, or from about 100 mW to about 500 mW. The irradiation laser power is 250 mW. The irradiation time for curing a single layer of the composition depends on the stereolithography method and may be adjusted appropriately. For example, in the DLP method, the irradiation time is about 1 to 60 seconds, but again depends on the specific formulation and method of stereolithography. These devices may be produced in an environment at about room temperature (e.g., 15 to 35° C.). The methods comprise the use of a laser with a laser speed from about 5 mm/s to about 150 mm/s, from about 10 mm/s to about 100 mm/s, or from about 25 mm/s to about 75 mm/s. In some embodiments, the laser speed is from about 5 mm/s, 10 mm/s, 20 mm/s, 25 mm/s, 30 mm/s, 40 mm/s, 50 mm/s, 60 mm/s, 70 mm/s, 75 mm/s, 80 mm/s, 90 mm/s, 100 mm/s, 120 mm/s, 125 mm/s, 140 mm/s, to about 150 mm/s, or any range derivable therein. The laser may also have a perimeter offset from about 10 μm to about 750 μm, about 50 μm to about 500 μm, or from about 100 μm to about 300 μm. In some embodiments, the perimeter offset is 10 μm, 20 μm, 25 μm, 30 μm, 40 μm, 50 μm, 60 μm, 70 μm, 75 μm, 80 μm. 90 μm, 100 μm, 125 μm, 150 μm, 200 μm, 250 μm, 300 μm, 400 μm, 500 μm, 600 μm, 700 μm, to about 750 μm, or any range derivable therein. The laser has a hatch spacing from about 5 μm to about 150 μm, from about 10 μm to about 100 μm, or from about 25 μm to about 75 μm. The hatch spacing is from about 5 μm, 10 μm, 20 μm, 25 μm, 30 μm, 40 μm, 50 μm, 60 μm, 70 μm, 75 μm, 80 μm, 90 μm, 100 μm, 125 μm, 150 μm, 200 μm, 250 μm, or any range derivable therein. In some embodiments, the laser comprises using a hatching offset from about 5 μm to about 500 μm, from about 25 μm to about 250 μm, or from about 100 μm to about 150 μm. In some embodiments, the hatching offset is 10 μm, 20 μm, 25 μm, 30 μm, 40 μm, 50 μm, 60 μm, 70 μm, 75 μm, 80 μm, 90 μm, 100 μm, 125 μm, 150 μm, 200 μm, 250 μm, 300 μm, 400 μm, 500 μm, 600 μm. 700 μm, to about 750 μm, or any range derivable therein.

The additive manufacturing techniques may comprise a chamber that has a temperature and the surface of the feedstock composition has a surface temperature when applied to the surface of the chamber. In some embodiments, the print surface temperature is less than about 200° C. 150° C. 100° C. 90° C., 80° C., 75° C., 70° C., 60° C., or about 50° C. Similarly, the chamber temperature is less than about 200° C. 150° C., 100° C., 90° C., 80° C., 75° C., 70° C., 60° C., or about 50° C.

The additive manufacturing methods may comprise applying one or more layers in the processing. In some embodiments, the manufacturing process comprises a number of layers from about 1 to about 10,000, from about 5 to about 1,000, or from about 10 to about 100 layers. The number of layers are from 1, 2, 5, 10, 25, 30, 40, 50, 60, 70, 80, 90, 100, 150, 200, 250, 500, 600, 700, 800, 900, 1,000, 1,250, 1,500, 2,000, 2,500, 3,000, 5,000, 6,000, 7,000, 8,000, 9,000, to about 10,000, or any range derivable herein.

After the stereolithography, optionally, a general secondary treatment, such as high-pressure mercury lamp irradiation, metal halide lamp irradiation, UV-LED irradiation, or heating, may be additionally performed. The secondary treatment can modify the surface after stereolithography, improve the strength, or accelerate curing. The secondary treatment can be performed in combination with stereolithography, although the secondary treatment is not necessarily required, depending on the stereolithography conditions.

In particular, when UV-LED irradiation is employed as a secondary treatment in the method of preparing a device described herein, any unreacted monomer contained in the AM parts can be cured by radicals generated from the photopolymerization initiator, which can further increase the mechanical strength.

In other aspects, the pharmaceutical compositions described herein may also be used in an additive manufacturing platform. Some of the additive manufacturing platforms that may be used herein include 3D printing such as stereolithography may be used to obtain the final pharmaceutical composition.

In some aspects, the pharmaceutical compositions described herein are processed in a final dosage form instead of as a device. The granules that are produced by the process may be further processed into a capsule or a tablet. Before formulation into a capsule or tablet, the granule may be further milled before being compressed into the capsule or tablet.

### IV. Definitions

The use of the word “a” or “an” when used in conjunction with the term “comprising” in the claims and/or the specification may mean “one,” but it is also consistent with the meaning of “one or more.” “at least one.” and “one or more than one.” As used herein “another” may mean at least a second or more.

As used herein, the terms “drug”, “pharmaceutical”, “active pharmaceutical ingredient”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, a reduction in the growth rate of cancer, or prevention of metastasis. Treatment of cancer may also refer to prolonging the survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1.2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide, and calcium hydroxide. Acceptable organic bases include ethanolamine, diethanolamine, triethanolamine, tromethamine, N-methylglucamine, and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified compound, wherein at least one of the compounds is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example, hydrochlorides, hydrobromides, sulfates, hydrogen sulfates or phosphates, salts with suitable carboxylic acids, such as optionally hydroxylated lower alkanoic acids, for example, acetic acid, glycolic acid, propionic acid, lactic acid or pivalic acid, optionally hydroxylated and/or oxo-substituted lower alkane dicarboxylic acids, for example, oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example, methanesulfonates, benzenesulfonates, p-toluenesulfonates or N-cyclohexylsulfamates (cyclamates).

The term “degradation” or “chemically sensitive” refers to a compound that is destroyed or rendered inactive and unacceptable for use. Degradation may include compounds which have one or more chemical bonds present in the compound has been broken.

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active agent in the composition is measured by powder x-ray diffraction.

A “poorly soluble drug” refers to a drug which meets the requirements of the USP and BP solubility criteria of at least a sparingly soluble drug. The poorly soluble drug may be sparingly soluble, slightly soluble, very slightly soluble or practically insoluble. In a preferred embodiment, the drug is at least slightly soluble. In a more preferred embodiment, the drug is at least very slightly soluble. As defined by the USP and BP, a soluble drug is a drug which is dissolved from 10 to 30 part of solvent required per part of the solute, a sparingly soluble drug is a drug which is dissolved from 30 to 100 part of solvent required per part of the solute, a slightly soluble drug is a drug which is dissolved from 100 to 1,000 part of solvent required per part of the solute, a very slightly soluble drug is a drug which is dissolved from 1,000 to 10,000 part of solvent required per part of the solute, and a practically insoluble drug is a drug which is dissolved from 10,000 part of solvent required per part of solute. The solvent may be water that is at a pH from 1-7.5, preferably physiological pH.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significance such as “significantly”) is not meant to imply statistical differences between two values but only to imply importance or the scope of the difference of the parameter.

Throughout this application, the term “about” is used to indicate that a value includes the inherent variation of error for the device, the method being employed to determine the value or the variation that exists among the study subjects or experimental studies. Unless another definition is applicable, the term “about” refers to +10% of the indicated value.

The term “homogenous” is used to mean a composition in which the components are mixed in such a way that the components are uniformly distributed amongst the composition. In a preferred embodiment, the composition is uniformly distributed in such a manner that there are no regions of a single component that are greater than 1 μm or more preferably less than 0.1 μm. In one embodiment, the composition is so homogeneously mixed in such a manner that there are no atoms of the electromagnetic energy absorbing excipients are adjacent to another atom of the electromagnetic energy absorbing excipients.

The terms “substantially” or “approximately” as used herein may be applied to modify any quantitative comparison, value, measurement, or other representation that could permissibly vary without resulting in a change in the basic function to which it is related.

A temperature, when used without any other modifier, refers to room temperature, preferably 23° C. unless otherwise noted. An elevated temperature is a temperature which is more than 5° C. greater than room temperature; preferably more than 10° C. greater than room temperature.

The term “unit dose” refers to a formulation of the pharmaceutical composition such that the formulation is prepared in a manner sufficient to provide a single therapeutically effective dose of the active agent to a patient in a single administration. Such unit dose formulations that may be used include but are not limited to a single tablet, capsule, or other oral formulations, or a single vial with a syringeable liquid or other injectable formulations. In some forms, the final pharmaceutical composition that is produced is no longer a powder and is further produced as a homogenous final product.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements and parameters.

Other objects, features, and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### V. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Preparation of Self Emulsifying Compositions Through Additive Manufacturing

### A. Material and Methods

i. Materials

Ritonavir (RTV) was obtained from TCI chemicals (Tokyo, Japan). Compritol 888 ATO (C888), Gelucire 44/14 (G44/14), Gelucire 48/16 (G48/16), and Gelucire 59/14 (G59/14) were all received from Gattefossé (Saint-Priest, France) as gift samples. Different molecular weights of polyethylene glycol (PEG) ranging from 400, 1000, 3350 and 6000 were purchased from Sigma (St. Louis, MO, USA) along with Tween 80 and Span 80. All other chemicals and reagents used in the study were of analytical grade obtained from Fisher Scientific (Waltham, MA, USA).

Human colon adenocarcinoma Caco-2 cells were purchased from the American Type Culture Collection (ATTC, Manassas, VA, USA). Dulbecco's modified eagle's medium (DMEM), Dulbecco's phosphate buffer saline (DPBS), Hanks' balanced salt solution (HBSS), 4-(2-hydroxyethyl)-1-piperazine ethanesulfonic acid (HEPES), fetal bovine serum (FBS), and trypsin-ethylenediaminetetraacetic acid (EDTA) were purchased from Grand Island Biological Company (Gibco, Billings, MT, USA). All other chemicals were of reagent grade and used without further purification.

TABLE 1
Composition of lipids used for formulation development.

| | | Melting | | |
|---|---|---|---|---|
| | | range | CMC | |
| Lipids | HLB | (° C.) | (mg/L) | Composition |
| Compritol 888 | 2 | 65-77 | N/A | Glyceryl dibehenate |
| ATO (C888) | | | | |
| Gelucire 44/14 | 11 | 42.5-47.5 | 72 ± 53 | Lauroyl polyoxyl-32 |
| (G44/14) | | | | glycerides |
| Gelucire 48/16 | 12 | 46-50 | 153 ± 31 | Polyoxyl-32 stearate |
| (G48/16) | | | | |
| Gelucire 59/14 | 14 | 57-62 | 40 | Lauroyl polyoxyl-32 |
| (G59/14) | | | | Glycerides (and) |
| | | | | PEG-150 |

### B. Screening of Surfactants and Lipids

i. Saturation Solubility in Lipids

Individual lipids and mixtures of different lipids were screened to evaluate the maximum saturation solubility of RTV. All the lipids and mixtures were kept on a shaking platform with the temperature maintained at 60° C. After the lipids were completely converted to their molten state, an excess of drug was added to them and let shake for 48 hours at 100 rpm. The samples were allowed to settle post shaking, and the supernatant was collected for analysis. The extraction was carried out using ACN and was analyzed using the HPLC method described in the later section. All the studies were performed in triplicates.

ii. Screening Lipid and Surfactant Mix

The lipids and mixture showing maximum saturation solubility, i.e., G48/16 and a 1:1 mixture of G48/16 and G44/14 were then screened with different surfactants to screen combinations that yield a solid product at room temperature. The solid state was evaluated visually, with semisolid pastes and viscous gels being rejected for further studies, as the end goal is to formulate solid granules. Briefly, 1:9, 1:4 and 1:1 mixture of PEG400, PEG1000, PEG3350, PEG6000. Tween 80 and Span 80 were prepared with G48/16 and 1:1 mixture of G48/16 and G44/14. These were then kept on a shaking platform with the temperature maintained at 60° C. After the mixtures were completely converted to their molten state, the systems were led to shake for another 1 hour and then taken off heating to cool to room temperature. The samples were collected form the cooled systems and evaluated for their state in three categories as semisolid paste, viscous gel, and solid mass.

iii. Phase Diagram Studies

The 4 mixtures selected from the screening process, namely (G48/16-PEG3350), (G48/16+G44/14 (1:1)-PEG3350), (G48/16-PEG6000), and (G48/16+G44/14 (1:1)-PEG6000) were then studied for their behavior with water using phase diagram studies. Phase diagrams were constructed following the general procedure outlined by Prajapati et al. For this purpose, mixtures ranging from 1:9 to 10:0 ratio of lipid to surfactant were prepared for the mixtures listed above in separate 200 mL flasks. Water was then added in 5% increments until 95% and then up to 99% to make up the volume up to ˜200 mL. Following the addition of water, each of the flasks were shaken for 15 mins in the 37° C. water bath for equilibration. Different phases were visualized by checking whether they were transparent, cloudy or phase separated, and then globule size analysis was conducted to determine sizes of oil globules formed during dilution with water in regions of phase diagrams having 80% or more water. In phase diagrams, there were three regions identified: (1) clear liquid phase that was viscous at the relatively low water content and formed microemulsion (ME) upon dilution to the higher water content. (2) cloudy liquid phase consisting of the emulsion (EM), and (3) the phase-separated (PS) region where the lipid mixtures partially separated from the aqueous phase to form a separate layer. All the studies were performed in triplicates. The phase diagrams were prepared and analyzed using Chemix software (Chemix School, Ver. 11.7, Bergen, Norway).

iv. Particle Size Measurements

A DLS Zetasizer (Malvern, UK) was used to measure particle sizes of selected lipid-surfactant mixtures at 80, 85, 90, 95 and 99% (w/w) water contents prepared for the construction of phase diagrams. Approximately 1 mL of sample was aliquoted from the mixture maintained at 37° C. and the particle size was determined at room temperature (25° C.). As there was a decrease in temperature during the transfer of samples from 37° C. water bath to the particle size analyzer; the determination of particle size made the data from different samples consistent to each other since they were determined at the same temperature. Mixtures having a consistent particle size under 100 nm and a polydispersity index (PDI) with a narrow distribution range were considered for further studies.

### C. Processing Space Design Optimization

i. Preliminary DSC and TGA Studies

Thermal analysis was carried out to understand the degradation temperature and thermal events of the components (G48/16, PEG3350, RTV, Physical mixture and the extruded granules) using thermogravimetric analysis (TGA) and differential scanning calorimetry (DSC). Briefly, a TGA/DSC 1 (Mettler-Toledo, Schwerzenbach, Switzerland) was used, and the samples were loaded into an open crucible, placed in the furnace, and heated from 35 to 350° C. at a rate of 10° C./min. The TGA was run under an ultra-purified nitrogen environment at a 50 mL/min purge gas flow rate. The data was collected and analyzed using the STAR software (Mettler-Toledo, Switzerland).

For the modulated DSC analysis, a Q20 DSC unit (TA® Instruments, New Castle, DE, USA) was used and 5-10 mg of the samples were weighed in T-zero aluminum pans (DSC Consumables Incorporated, Austin, MN, USA) using a calibrated microbalance. The samples were heated from 35 to 150° C. at a 5° C./min ramp rate and the temperature was modulated by 1.00° C. every 60 s, under a nitrogen flow of 50 mL/min. The samples were also subjected to a heat-cool-heat cycle with the first heating cycle at 5° C./min from 35° C. to 150° C., cooled at 10° C./min to 0° C. and then heated again at 5° C./min to 150° C. The collected data were analyzed and plotted as temperature (° C.) versus heat flow (W/g).

ii. Hot-Met Extrusion (HME)

The processing parameters were selected based on thermal investigation and the DOE design. Granules were prepared at 40% drug (RTV) loading in a 60 matrix of 9:1 G48/16 to PEG3350 ratio. The extrusion was carried out using a Leistritz ZSE 12 HP-PH 12 mm twin screw co-rotating HME extruder (Leistritz Advanced Technologies Corp., Nuremberg, Germany). The optimized temperature profile and the screw design used for the extrusion process based on the thermal investigation are shown in FIG. 7. The blend was introduced into the feeding section of the extruder using a calibrated volumetric feeder (Brabender twin screw feeder with stirring agitators, Brabender Technologie, Mississauga, ON, Canada) at a feeding rate of 3 g/min. The granules collected post-extrusion were stored in a validated desiccator for further use and characterization.

iii. DOE Study for Processing Parameters

In order to optimize the effect of extrusion parameters on the formulated granules and to understand which parameters have the most significant impact on the granule properties, a A randomized 2 factor L-optimal response surface design was built using Design Expert 10. The two independent variables were the processing temperature and screw speed. Extrusion temperature and extrusion speed were varied between a minimum and maximum value. Extrusion temperature was varied between 25 and 130° C. while the extrusion speed was varied between 5 and 50. The responses studied include dispersion time and angle of repose. A quadratic model was fit for both responses. The model was considered significant if p<0.05 and lack of fit was not significant. Table 3 contains a detailed list of all the runs performed as a part of the DoE.

iv. Measurement of Dispersion Time

The granules processed using the runs from the Design Expert software were added to a beaker with 100 ml of water under constant stirring at 25 rpm. The time taken for the granules to disperse to form clear to translucent regions was noted as the dispersion time.

v. Angle of Repose Measurements

The angle of repose is the angle formed by the horizontal base of the bench surface and the edge of a cone-like pile of granules. A stainless steel funnel was used to drop the granules for analysis. The size of the orifice was 10 mm, and the funnel was fixed in place, 4 cm above the bench surface. After the cone from 5 g of sample was built, height (h) and radius (r) of the granules forming the cone were measured. The angle of repose (0) was calculated as follows:

$$θ = {tan}^{- 1} ( h / r ) \qquad ( 4.1 )$$

### C. Formulation Characterization

i. Powder X Ray Diffraction (PXRD)

The crystallinity of RTV in the formulated granules was evaluated using a benchtop Rigaku MiniFlex 600 (Rigaku Corporation, Tokyo, Japan). The instrument (equipped with a Cu Kα radiation source) was set to 15 mA with a 40 kV voltage. The samples (G48/16, PEG3350, RTV, Physical mixture and the extruded granules) were evenly spread into the holder and analyzed over a 2θ range of 5-60° with a scan speed of 2°/min and a step size of 0.02°/min.

ii. FTIR

The intermolecular interactions between RTV and the carrier matrix were investigated using an FTIR analysis (iS50 FTIR equipped with a SMART OMNI-Sampler, ThermoFisher Scientific, Waltham, MA, USA). The samples (G48/16, PEG3350, RTV, Physical mixture and the extruded granules) were analyzed from 4000-700 cm⁻¹, at a resolution of 4 cm⁻¹ for % transmittance with 64 scans per run. The background was collected before every run. Weak intermolecular interactions were analyzed and assessed on the OMNIC™ series software (ThermoFisher Scientific, Waltham, MA, USA) for the collected spectra.

iii. Polarized Light Microscopy (PLM)

The RTV loaded granules along with the raw materials were evaluated for the presence of crystallinity using an Olympus BX53 polarized photomicroscope (Olympus America Inc., Webster, TX, USA). The microscope was equipped with a Bertrand lens. The birefringence in crystalline substances was observed under 10× magnification. The images captured using a QICAM Fast 1394 digital camera (QImaging, Surrey, BC, Canada) with a 530-nm compensator (U-TP530, Olympus® Corporation, Shinjuku City, Tokyo, Japan) were analyzed using Linksys 32 software® (Linkam Scientific Instruments Ltd., Tadworth, UK).

iv. Scanning Electron Microscopy (SEM)

The morphology of the formulated granules was analyzed by scanning electron microscopy (SEM). The powders were mounted on SEM stubs and sputter-coated with 12 nm of platinum/palladium (Pt/Pd). Imaging was performed using an FEI Quanta650 ESEM instrument (FEI, Hillsboro, OR).

v. Solid-state Nuclear Magnetic Resonance (ssNMR)

All experiments involving solid-state NMR were conducted using a Bruker Avance III HD 400 spectrometer from Bruker BioSpin (Billerica, Massachusetts, USA) operating at a proton frequency of 399.87 MHz. In each case, spectra were obtained utilizing a Bruker 4 mm HFX magic-angle spinning (MAS) probe configured for double resonance, and tuned to both 1H and ¹³C frequencies, while maintaining a MAS frequency of 12 kHz at a temperature of 298 K. The samples were securely placed in 4.0 mm rotors for all experimental runs, and the standard pulse lengths for ¹H and ¹³C were 3 μs and 5 μs, respectively. For the 1D 13C-1H cross-polarization (CP) MAS experiments, a radio-frequency strength of 62.5 kHz was employed for both channels during the polarization transfer. To ensure effective polarization transfer, a linear ramp was applied to the proton channel, starting from 50% and reaching 100%. Additionally, 1H heteronuclear decoupling was achieved using a SPINAL-64 scheme at a frequency of 83.3 kHz during the ¹³C detection. All experiments were executed with an acquisition time of 24.5 ms. All data underwent processing through Bruker TopSpin software, and the referencing was performed externally, using the alpha-glycine carbonyl peak at 176.49 ppm as the reference point.

v. Rheological Measurements

The rheological properties as a function of temperature for the drug, lipid and formulation were investigated using a Discover HR-2 Hybrid rheometer. All the measurements were conducted from 25° C. to 80° C. at a ramp rate of 10° C. using parallel plates with an 8 mm diameter geometrical setup. The flow curves of each material, i.e., the shear viscosity versus the temperature were obtained over a shear rate range of 1 s⁻¹.

vi. Preparation of Self-Emulsifying Granules Using Conventional Melt-Mixing Process

The components for formulating granules were weighed into an open beaker and melted on a hot plate until it formed a clear solution. The mixture was heated at 80° C. to ensure complete mixing of lipids and dispersion of the drug in the same. All samples were mixed using a high speed paddle mixer for 5 mins in the molten state to obtain homogeneous mixing of the components. Samples were then allowed to solidify at room temperature and stored for at least 48 h before analysis.

vii. Direct Powder Extrusion (DPE) 3D Printing

A pneumatic extrusion-based bioprinter (BIOX, Cellink, Gothenburg, Sweden) was used to 3D print the physical mixtures and the formulated granules. Cylindrical tablets with a diameter of 8 mm and tablet height of 5 mm with 100% rectilinear infill were printed at a printing speed of 5 mm/s using a 20-gauge needle. The print pressure was kept at 150 kPa.

viii. Dissolution Testing

Dissolution testing of 40% RTV loaded self-emulsifying granules against pure drug and the physical mixture was conducted using water, pH shift setting and FaSSIF (Biorelevant.com, London, UK). The dissolution tests were performed in an Agilent dissolution apparatus using the 200 mL vessels and their paddles. Dissolution tests were conducted by adding the equivalent of 100 mg of ritonavir drug content into 150 mL of FaSSIF or buffer pH 6.5 media at 37.0±0.5° C. and a paddle speed of 100 rpm. The sample points were measured at 5, 10, 15, 30, 60, and 120 min. 30% drug loaded granules were also formulated using the optimized process and used as a performance comparator against 40% drug loaded granules.

The pH-shift dissolution tests for 40% RTV loaded self-emulsifying granules against pure drug and the physical mixture were performed using the same equipment in two stages. First, 100 mg of RTV loaded granules were added to 30 mL of HCl 0.01 M (pH 2.0) for 30 min. Thereafter, 120 mL of phosphate buffer 0.01 M (pH 6.8) was added into the vessel, completing a volume of 150 mL, and the samples were taken at the same time points of the previously described dissolution test.

For analysis using FaSSIF, the pH-shift dissolution tests was performed using the same equipment in two stages again. 100 mg of RTV loaded granules were added to 30 mL of HCl 0.01 M for 30 min. Thereafter, 120 mL of FaSSIF was added into the vessel, completing a volume of 150 mL, and the samples were taken at the same time points of the previously described dissolution test.

When recollecting the samples, they were passed through polyethersulfone 0.2 μm filters. Then, 0.5 mL of the samples were mixed with 0.5 mL of acetonitrile for HPLC analysis. The samples were measured at 240 nm using a Vanquish HPLC system (Thermo Fisher Scientific Inc., Sunnyvale, CA, USA) with a Avantor ACER EXCEL 3 C-18-AR column (250×2.1 mm, 2 μm particle size) (VWR International, Radnor, PA, USA) at a flow rate of 1.5 mL/min. Two mobile phases were used. The mobile phase A was potassium monophosphate buffer (pH adjusted to 4) and the mobile phase B was acetonitrile (Thermo Fisher Scientific Inc.). They were mixed in a 45:55 ratio.

RTV loaded granules prepared using the conventional process were tested against extruded granules using the pH shift method to understand the effect of processing method on the formulation. Tablets prepared using the drug loaded granules using direct powder extrusion 3D printing were the compared to tablets prepared using only the physical mixture through DPE 3D printing to compare the performance of granules and physical mixtures for the printing process.

### D. Permeability Studies

i. Cell Lines and Culture Conditions

Caco-2 cells were used for the in vitro cellular experiments. These cells were cultured in DMEM, supplemented with 10% FBS and 1% penicillin/streptomycin, and incubated at 37° C. with a 5% CO₂ atmosphere. Subsequently, cells were split using 0.25% trypsin-EDTA at 70-80% confluency, and the media was replaced every 2-3 days.

ii. MTT Assay

The cell viability of the SMEDDS in the Caco-2 cells was determined in a 96-well plate using the Cell Proliferation Assay kit (Sigma-Aldrich, Saint Louis, MO, USA) containing 3-(4, 5dimethylthiazol-2-yl)-2, 5-diphenyltetrazolium bromide (MTT) according to the manufacturer's instructions. Briefly, the cells were seeded at a density of 2×10⁴ cells per well in a 96-well plate and incubated for 24 h at 37° C. The cells were washed twice with HBSS-HEPES buffer (pH 7.4) and then treated with 100 μL of SMEDDS, diluted with HBSS-HEPES buffer (pH 7.4) at different concentrations of ritonavir ranging from 10 to 500 μg/mL, for 4 h at 37° C. Subsequently, 10% MTT solution was added to each well and incubated for 4 h at 37° C. Finally, the absorbance was read at 570 nm using a multi-well plate reader (Bio Tek, Winooski, VT, USA). The cell viability (%) was calculated as follows:

$$Cell viability [ \% ] = \frac{{OD}_{570 ( sample )} - {OD}_{570 ( blank )}}{{OD}_{570 ( control )} - {OD}_{570 ( blank )}} \times 100 \qquad ( 4.2 )$$

where OD is the optical density.
iii. Caco-2 Permeability Testing

To evaluate the permeability profiles of the ritonavir solution and the SMEDDS across the Caco-2 monolayers, in vitro permeability studies were performed using a previously well-established method (Silva et al., 2018). Briefly, Caco-2 cells (3×10⁵ cells per well) were seeded in 12-well-Transwell® inserts (1.12 cm², 0.4 μm pore size; Corning Inc., Corning, NY, USA) and grown for 21 days to form a differentiated confluent monolayer. DMEM medium was replaced every subsequent day. An epithelial volt-ohm meter [Millicell® ERS-2 (Electrical Resistance System); EMD Millipore Corporation, USA] was used to measure the transepithelial electrical resistance (TEER), according to the manufacturer's instructions. The cell monolayers that exhibited initial TEER values of >500 Ω·cm² were used during this permeability experiment, and the TEER values of the Caco-2 cell monolayers were monitored before and after each transport experiment. The studies were performed from the apical to the basolateral direction in the HBSS-HEPES buffer (pH 7.4). The apical and basolateral chambers of the Transwell® were equilibrated with 0.5 mL and 1.5 mL of prewarmed HBSS-HEPES buffer (pH 7.4) for 30 min at 37° C., respectively. After equilibration, the medium of the apical chamber was replaced with 0.5 mL of ritonavir solution or ritonavir loaded SMEDDS (equivalent to 50 μg/mL ritonavir), and incubated at 37° C. Subsequently, a total of 500 μL of the sample was withdrawn from the basolateral chamber at predetermined time points (0.5, 1, 2, 3, and 4 h) and replenished immediately with an equal volume of prewarmed HBSS-HEPES buffer (pH 7.4) to retain a constant volume. The collected samples were then analyzed using HPLC.

The apparent permeability coefficient (Pₐₚₚ) and enhancement ratio (ER) were calculated from the linear parts of the permeation curves, as described by Hubatsch et al., The Pₐₚₚ was calculated as follows:

$${P}_{app} = \frac{dQ}{dt} \times \frac{1}{A \cdot {C}_{0}} ( cm \cdot {s}^{- 1} ) \qquad ( 4.3 )$$

where dQ/dt, A and Cₒ are the steady-state flux (μmol/s), the surface area of the monolayers (cm²), and the initial concentration in the donor chamber (μM), respectively. Finally, the ER was calculated using the ratio of the Pₐₚₚ value of each formulation to that of the control.

### E. Results and Discussions:

i. Preformulation Studies

Conventional self-emulsifying systems are available in liquid form as most of the excipients used are liquid in nature. Although these liquid systems have good qualities of delivering therapeutics, they lack good storage stability and tend to degrade in presence of external stimuli such as light, heat, moisture, air and change in pH. These stability issues can be circumvented in the case of solid self-emulsifying systems as the dispersion of the active moiety in the solid matrices would lead to retardation of the rate and extent of degradations. Also, in the case of manufacturing and handling, solid state systems are easier to handle, making their manufacturing more feasible and cost effective from a scale-up point of view. These solid systems can be processed as granules which can then be used for making oral compressible tablets or filling into capsules for oral delivery. These can also be used with the upcoming 3D printing setup for personalizing drug delivery to patients. Use of uniformly dispersed drug in homogenously formulated granules would lead to better drug distribution in case of 3D printed tablets as compared to use of physical mixture for direct powder extrusion.

The first step in selection of such carrier system is the choice of lipid carriers. Solubility of the drug in these lipid carriers will determine the drug loading of the system thus affecting the final dosage form size based on the drug dose. The aim of the study was to develop a carrier platform that could self-emulsify the loaded drug post administration in the GIT, leading to improved solubility and permeability, especially in case of BCS Class II and IV drugs. The carrier matrix which would show the highest saturation solubility would be selected for further studies. Non-ionic lipids are reported to be less toxic compared to their ionic counterparts and thus were selected for this study. After measuring the solubility of RTV in the lipid matrices, it was seen that G48/16 and a 1:1 mixture of G48/14 and G48/16 showed the highest solubility values. These lipids systems amongst the others were then selected for their screening with surfactants. Although the selected gelucires also act as non-ionic surfactants, their inherent solid state at ambient condition despite the lipidic composition was of interest for formulation development. Additional surfactant was screened for adding to the formulation to aid the dispersion of the lipid based carrier system for faster self-emulsification properties.

The screening of the selected lipid systems, G48/16 and a 1:1 mixture of G48/14 and G48/16 was conducted across a range of surfactants. The end goal was to have a mixture that remained in its solid state under ambient conditions. The lipid systems and surfactants were mixed in a 1:9, 1:4 and 1:1 ratio. Post melt-mixing and cooling down to ambient condition, the mixtures that remained as a semisolid paste or a viscous gel were rejected and the mixtures that remained as a solid mass were selected for further studies. As seen in Table-2, mixtures of the lipid carrier systems with PEG 400, PEG 1000, Tween 80, and Span 80 resulted in semisolid pastes or viscous gels. Only resulting mixtures using PEG3350 and PEG6000 were solid at ambient conditions. These four combination matrices were then selected for further studies, to then narrow down and select one system based on the particle size distribution post dispersion in water using phase diagram studies.

TABLE 2
Lipid-Surfactant mixture state screening

| | With G4816 | | | With G4414 + G4816 (1:1) | | |
|---|---|---|---|---|---|---|
| Surfactants | 1:9 | 1:4 | 1:1 | 1:9 | 1:4 | 1:1 |
| PEG 400 | Semisolid paste | Viscous gel | Viscous gel | Semisolid paste | Semisolid paste | Viscous gel |
| PEG 1000 | Semisolid paste | Viscous gel | Viscous gel | Semisolid paste | Semisolid paste | Viscous gel |
| PEG 3350 | Solid | Solid | Solid | Solid | Solid | Solid |
| PEG 6000 | Solid | Solid | Solid | Solid | Solid | Solid |
| Tween 80 | Semisolid paste | Viscous gel | Viscous gel | Semisolid paste | Viscous gel | Viscous gel |
| Span 80 | Semisolid paste | Viscous gel | Viscous gel | Semisolid paste | Viscous gel | Viscous gel |

The way these self-emulsifying systems disperse, and form emulsions largely depends on the physicochemical properties of excipients, their concentration, and the overall composition of the system. According to some researchers, SNEDDS form nano emulsion spontaneously with a droplet size of less than 100 nm whereas; SMEDDS form micro emulsion with the globule size in the range of 100-250 nm. Although, some authors differentiate SMEDDS as a transparent and translucent emulsion compared to SEDDS, these terminologies are widely used interchangeably by many researchers. Most authors have suggested using SNEDDS terminology instead of SMEDDS, as the emulsion formed after dilution is not thermodynamically stable and displays properties similar to nanoemulsions.

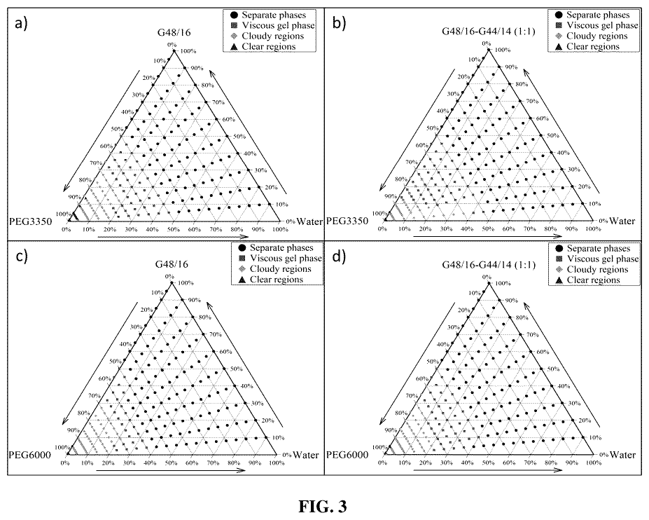

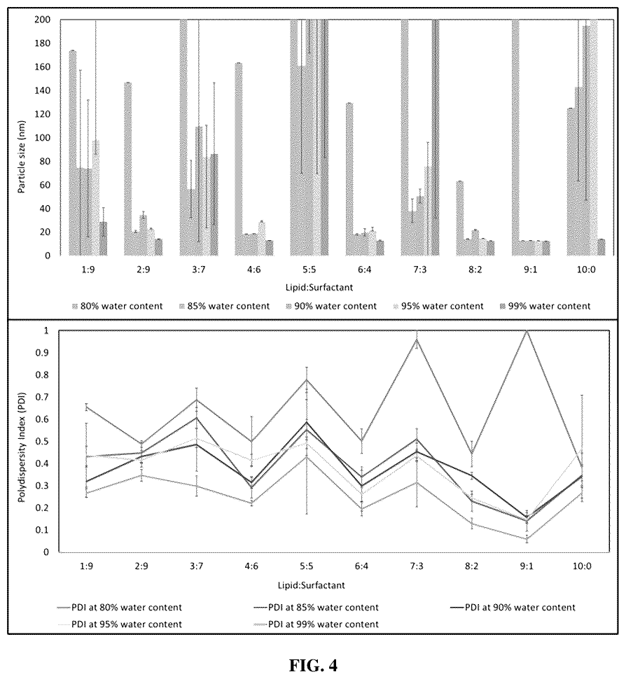

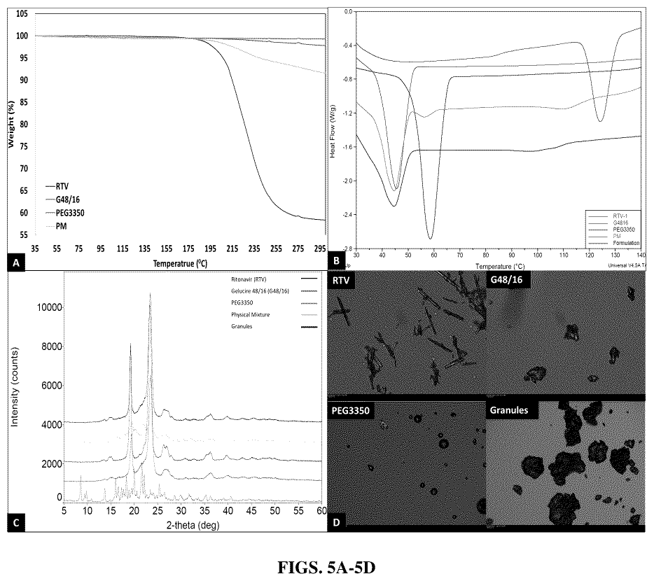

FIG. 3 shows ternary and pseudo ternary phase diagrams of Gelucires (G48/16 and G48/16+G44/14 (1:1)) along with PEG (PEG3350 and PEG6000) and water, respectively, where each apex of the diagram represents 100% of the labeled component. The mixtures combinations of (G48/16-PEG3350), (G48/16+G44/14 (1:1)-PEG3350), (G48/16-PEG6000), and (G48/16+G44/14 (1:1)-PEG6000) all showed a larger region of distinct separate phases (as seen by the black region in the phase diagram) where the shake times and the amount of water were not enough to disperse the phase and create a nanoemulsion. As the amount of water increased, the mixtures dispersed in the water with first forming a viscous gel phase (red region in the phase diagram) with a boundary layer around it. The mixtures then resulted in forming a cloudy microemulsion as seen by the green region in the phase diagrams, which further transformed into a narrow blue zone representing the nanoemulsion. G48/16-PEG3350 had the widest region that formed a nanoemulsion and dispersed faster than the other systems. Clear systems were seen only when the lipid to surfactant ratio was below 2:8 with G48/16. As compared to PEG6000, the use of PEG3350 formed clear solutions at all lipids to surfactant ratios with G48/16-G44/14 (1:1), whereas the use of PEG6000 as a surfactant did not form clear systems. Also, G48/16-PEG3350 formed a clear whitish solution with time for complete dispersion less than 5 mins and no phase separation post 48 hrs. On the other hand, mixture of G48/16 with PEG6000, formed a translucent solution. It took 13 to 20 mins for the system to completely disperse with precipitation seen post 48 hrs which later dispersed on shaking. G48/16+G44/14 (1:1)-PEG3350 formed clear whitish solution with 5 to 10 mins for complete dispersion. Phase separation was seen with the lipid crashing out in less than 24 hours, which then dispersed post stirring. G48/16+G44/14 (1:1) mixture with PEG6000 formed a translucent solution indicating the formation of microemulsion that took 20 mins for complete dispersion with phase separation in less than 24 hours, that dispersed after stirring. It can be said that PEG3350 performs better than PEG6000 to disperse and maintain the emulsified system along with G48/16 over the lipid mixtures system.

Particle size measurements showed a wide dispersion across the range of different mixture ratios and dilutions. Absence of surfactant in the mixture resulted in a higher particle size as compared to mixtures with addition of surfactant. Particle sizes measured at 99% water dilutions showed the lowest values across all ratios of lipid to surfactant mixtures. The distribution of particle sizes was the lowest starting at 85% water dilution across all lipids to surfactant ratios except 1:9 mixture. All of the dilutions are in the nanoemulsion range and the 9:1 mix had the lowest distribution and hence was selected for optimizing the extrusion process and further studies for formulation and process development.

TGA and DSC were used to understand the degradation temperatures limits for RTV, and the excipients involved, and to understand the thermal events to set the limits for the process optimization space. DSC revealed that the excipients had melting points below the melting point of RTV, with the drug melting around 127° C. TGA shows that the drug and the excipients are stable and do not degrade below 150° C. These values were used to set up the limits for the DOE study with the lower processing temperature being at ambient conditions or 25° C. and the maximum processing temperature being near the drug melting point, that is 130° C., which ensures complete melting and mixing of the involved drug and excipients.

ii. Hot-melt extrusion of granules and process optimization

HME is a continuous pharmaceutical process that involves conveying of polymeric materials with a rotating screw at temperatures above their glass transition temperature (T_{g}) and sometimes above the melting temperature (Tₘ) to achieve molecular level mixing of the APIs and carriers. This molecular level mixing converts the components into an amorphous product with a uniform shape and density, thereby increasing the dissolution profile of the poorly water soluble drug. Also, good content uniformity can be achieved for very low drug loading systems due to intimate mixing of components.

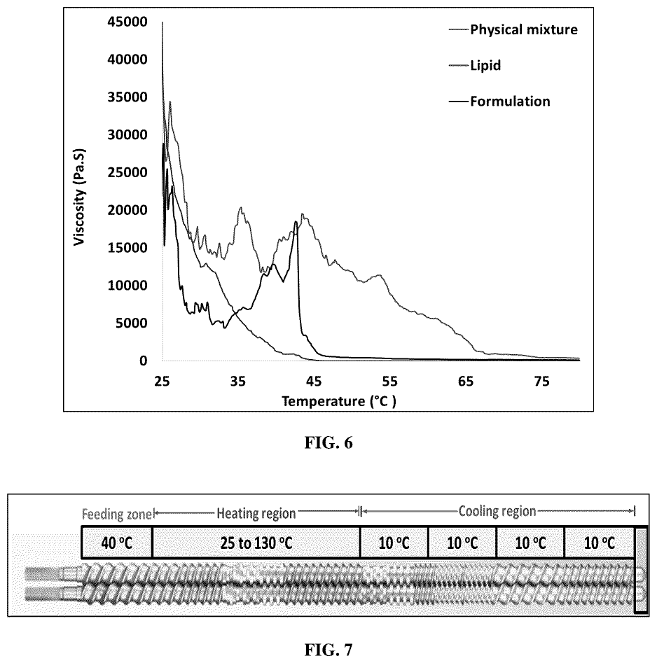

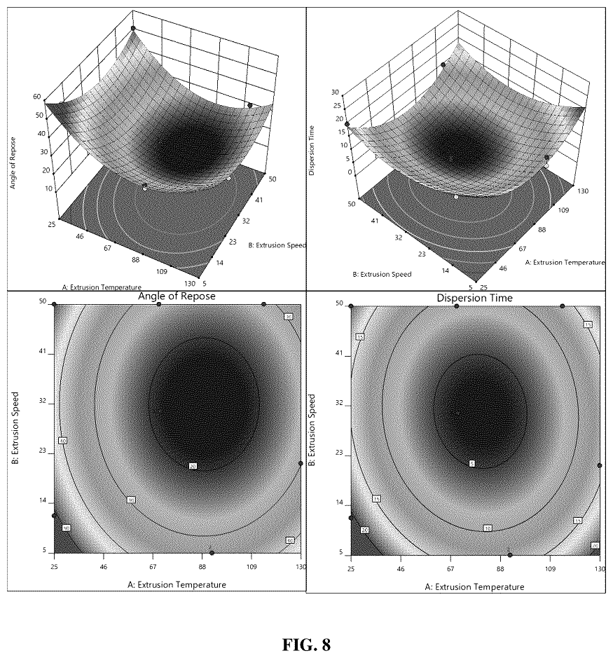

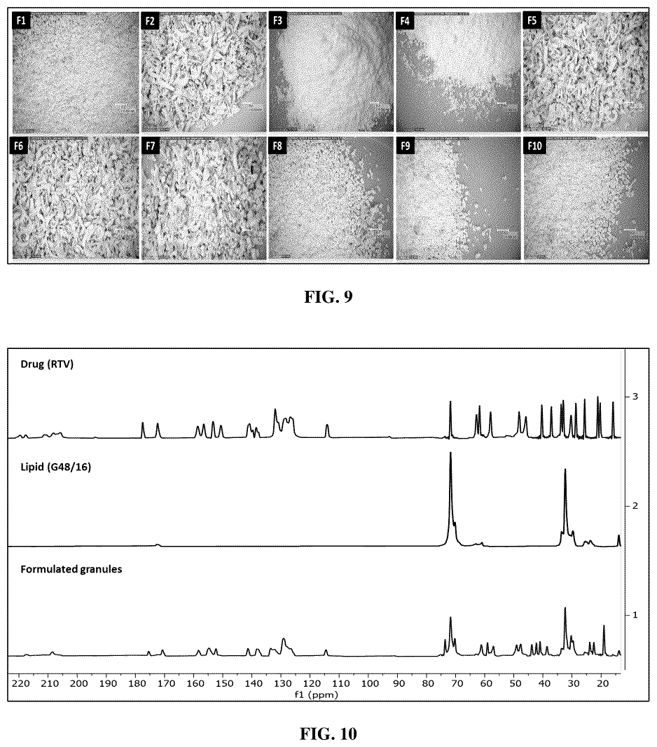

The rheological properties of the lipid, physical mixture and formulation were accessed to optimize the processing conditions for the printing process. As can be seen from FIG. 6, the viscosity of the three materials generally decrease as a function of temperature, this is attributed to the increase in molecular motion and kinetic energy in the molecules. This allows easier processing at temperatures closer to 70° C. The temperature ramps cycle for these materials overlaps with the melting processes. consequently, there are a few peaks visible during the cycle. It was interesting to see that viscosity increases at temperatures between 35 to 45° C. and this is believed to be indicative of the formation of structures that resist flowing.

The working range constraints for DOE were set based on the data from the thermal analysis, which kept the lower end at room temperature and the higher end at 130° C. to ensure complete mixing of all the involved entities. The processing speed were set from 5 rpm to 50 rpm. Granules were processed using the 10 different processing conditions based on DOE and the dispersion times and angle of repose was evaluated for each.

At low extrusion temperatures and low extrusion speed, the angle of repose and dispersion time was high. The same trend followed at high extrusion speeds and low extrusion temperatures. However, when the extrusion temperature and speed were maintained in the mid-range between 60-90° C. for extrusion temperature and between 20-45 for extrusion speed, the dispersion time and angle of repose were significantly lower. Interestingly, the dispersion time and angle of repose were negatively impacted by high extrusion temperatures when combined with low or high extrusion speed.

The extruder's screw consisted of a conveying system and mixing zones which helped in transport and efficient mixing. The first feeding zone served the purpose of pre-heating the feed and warming the drug-excipient feed close to the lipid carrier's melting temperature. This was followed by the heating region which consisted of mixing elements to ensure molecular level dispersion of the drug in the carrier system. An increase in temperature probably caused a higher distribution of the lipid constituents in the solid matrix due to a decrease in the melt viscosity. Using a cooling system for the extruder the temperature was brough down for the zones following the heating zone to convert the molten feed to its solid state and break down into granules. Solid-state analysis of the processed granules showed that the drug was converted to tis amorphous state and showed absence of crystallinity in the processed system. The presence of crystalline peaks seen in the formulation plots in case of XRD analysis, correspond to the peaks associated with G48/16 and PEG3350. The peaks characteristic to RTV are absent in the formulation.

The formulation chosen for further studies had 30% and 40% drug loading to evaluate the efficiency of our carrier system and processed at 30 rpm with heating zone set to 70° C. The drug was miscible in the lipid system and thus was loaded in its amorphous state post melt mixing and quench cooling in the extruder.

TABLE 3
Design of Experiment runs to understand the
effect of processing space on granules.

| | Factor 1 | Factor 2 | Response 1 | Response 2 |
|---|---|---|---|---|
| | A: Extrusion | B: Extrusion | Dispersion | Angle of |
| Run | Temperature | Speed | Time | Repose |
| F1 | 69.63 | 50.00 | 6 | 23.78 |
| F2 | 114.25 | 50.00 | 15 | 29.74 |
| F3 | 25.00 | 50.00 | 20 | 50.65 |
| F4 | 25.00 | 11.75 | 20 | 50.44 |
| F5 | 130.00 | 21.20 | 13 | 28.74 |
| F6 | 92.20 | 5.00 | 15 | 33.25 |
| F7 | 92.20 | 5.00 | 12 | 34.93 |
| F8 | 70.15 | 30.65 | 4 | 20.27 |
| F9 | 70.15 | 30.65 | 5 | 18.43 |
| F10 | 70.15 | 30.65 | 4 | 18.97 |

TABLE 4
Fit statistics for conducted DoE runs.

Response

| Fit statistic | Angle of repose | Dispersion time |
|---|---|---|
| R2 | 0.9864 | 0.9487 |
| Adjusted R2 | 0.9694 | 0.8845 |
| p-value | 0.0008 (significant) | 0.0109 (significant) |

The main limitation of HME is that a higher energy input is required to produce products compared to other techniques and may exclude some thermolabile compounds due to high processing temperatures (Kallakunta et al., 2019). However, in the case of lipidic excipients, there is no need for high-energy input since these carriers have relatively lower melting points compared to polymeric excipients (Prajapati et al., 2012).

Subsequently ssNMR was used to analyze the intermolecular interactions between the drug, lipid, and the final formulation. The appearance and disappearance of new peaks indicates intermolecular interactions in the drug lipid-based mixtures. It was observed that there was a new peak at 73.56 ppm in the spectra for the formulation and the original peak in neat drug at 150, 37 disappeared in formulation spectra. Apart from this there was an upward shift in the peaks at 177.29 and 172.54 in the formulation spectra. These findings corroborate some of the initial FTIR findings indicating the intermolecular interactions and its impact on drug crystallinity and stabilization.

iii. Performance Testing of Granules

Solid self-emulsifying systems work on the same principle as liquid-SEDDS, by generating emulsion when exposed to GI fluid with mild agitation (Panigrahi et al., 2018; Upadhyay et al., 2013). Bioavailability of hydrophobic drugs is affected by their poor dissolution rate under aqueous environment. These drugs administered in a self-emulsifying system reside in the emulsion droplets rather than coming in direct contact of GI fluid and release of drug from SEDDS is explained by the partitioning of drug between the hydrophobic and the aqueous layer (Lipid-Based Formulations A Winning Strategy to Overcome Oral Bioavailability Challenges People Make Our Name). In comparison to the normal emulsion, the droplet size of SEDDS is smaller which provides higher interfacial surface area for drug release and improves the dissolution of the lipophilic drugs. Improved dissolution results in a higher rate and extent of absorption and more reproducible plasma concentration (Meirinho et al., 2022; Ameta et al., 2023; Upadhyay et al., 2023).

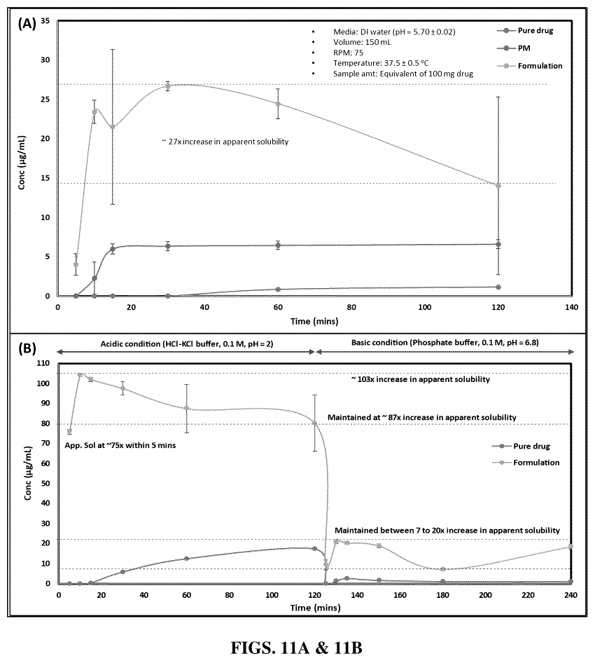

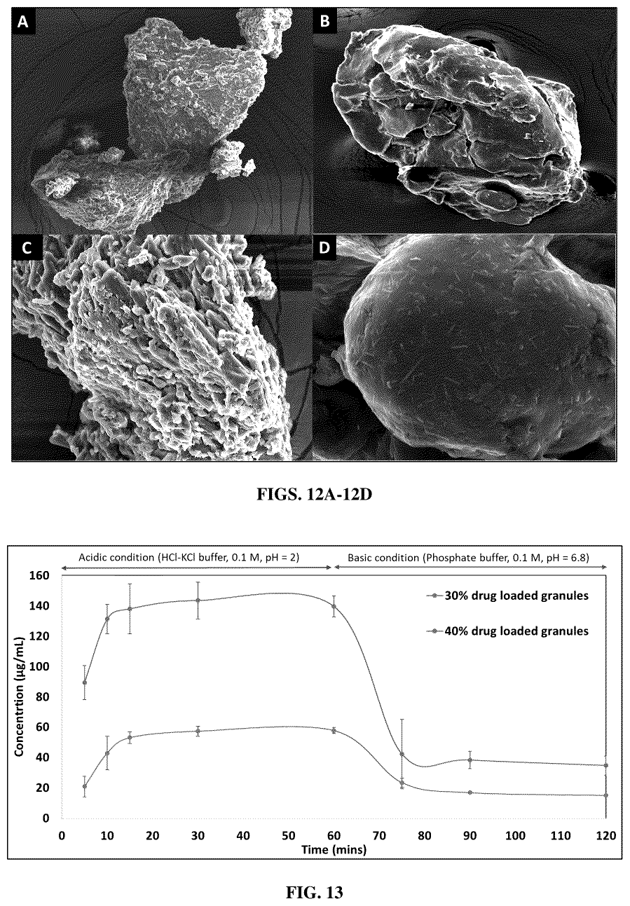

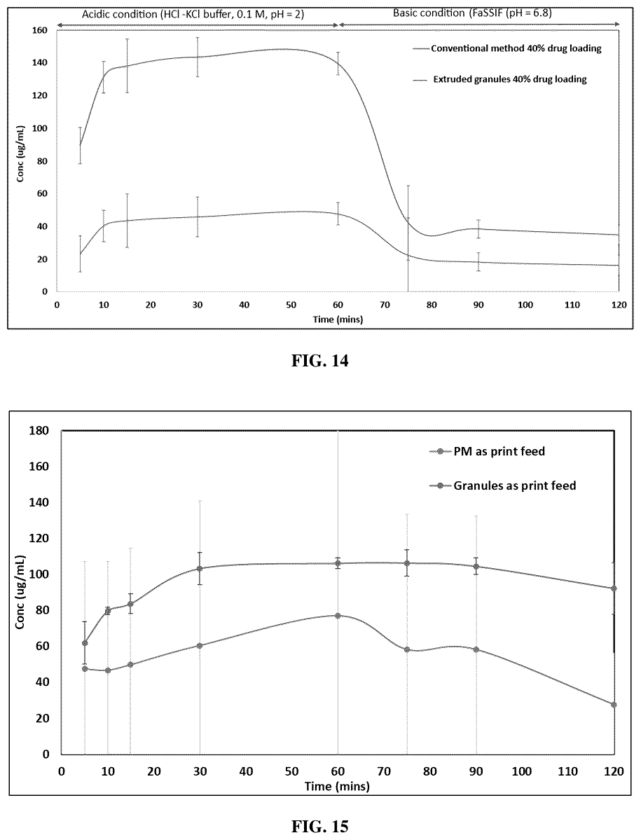

Initial studies to understand the inherent capability of the carrier system to improve the drug solubility were conducted using DI water with no media additives. 40% drug loaded granules performed better than the physical mixture and showed higher concentration as compared to the PM and pure drug. This was indicative of the role of melt mixing carried out using extrusion to create a homogeneously dispersed self-emulsifying matrix of lipid and surfactant that was able to improve the drug's apparent solubility. DI water had a pH around 5.3 which could be the reason behind the downward transition of the release profile seen for the formulation in FIG. 11A. To better understand the pH dependency of the drug and the formulation the studies were then conducted using buffers in a pH shift manner.

Both the pure drug and the formulation had increased concentration in acidic media post release which decreased drastically as the media was shifted to a basic pH. This pKa dependent solubility of the drug resurfaces itself in case of the formulation as well, which indicates crashing out of the drug in basic pH. In acidic conditions, the formulation had approximately 75-folds increased apparent solubility as compared to the pure drug, which achieved a peak maximum of 103-fold increase before falling down to the lower end of 7-fold increase apparent solubility in basic pH. In comparison to crystalline RTV dissolution profile, the granules demonstrate a significant increase in both rate and extent of dissolution in the initial stage, in addition to sustaining the supersaturated state over an extended period (i.e., 120 min) as it did not revert to crystalline state. The drop in the release was seen post change in the pH of the system.

30% and 40% drug-loaded granules were prepared using the optimized processing parameters from DOE. These were then compared using Sem for their surface morphology and for their release performance. SEM was carried out to understand the surface morphology of 30% and 40% drug loaded granules. 30% granules were formulated as a control comparator value under the supersaturation levels of observed solubility values in the lipid matrices. 40% drug loading was selected to make sure we run the system over the supersaturation levels to understand the performance of our carrier system and its ability to maintain increased hypothesized solubility levels. Granules in both the carrier systems show a rough surface morphology. In the case of 30% drug loaded system the drug is solubilized in the carrier matrix and no unsolubilized drug crystals can be observed on the granule surface. Whereas a close look at the 40% drug loaded granules clearly shows the presence of needled shaped RTV drug crystals on the surface of the granules, indicative of unsolubilized drug.

RTV shows a pH dependent solubility profile based on its pKa. Both the formulations show an increased apparent solubility based on the release profile which then decreases drastically as the pH shifts from acidic to basic media. 40% drug loading shows a higher increase in apparent solubility which might also be due to the ease of dispersion and solubilization of the drug present on the surface which can be seen in the SEM analysis. In spite of decrease in the concentrations post pH shift, the increased drug load formulation is able to maintain a higher concentration in the system.

iv. Comparison of Extruded Granules Against Conventionally Prepared SNEDDS.

One of the aims of this study was also to compare the performance of the HME process to the conventional melt mixing or melt fusion process of formulating lipid based drug delivery systems. The fusion method produced granules from a congealed mass. The efficient and intense mixing while extruding the material inside the barrel was able incorporate more API particles into the lipid matrix and make a formulation with a better and more uniform release behavior compared to heat fusion (Repka et al., 2018). The reason for this might be the breaking down of lipid chains into shorter moieties and efficient distribution of PEG in between these lipid chains to ensure uniform dispersion post contact with the aqueous media (Suk et al., 2016). Formulations prepared by the method showed an initial burst release which can be attributed to presence of needle shaped drug crystals in the surface of the granules as can be seen from the SEM images in FIG. 12. The stirring force was not as efficient as shearing force by HME, where RTV could be completely embedded within the matrix along with molecular dispersion of Peg in the G48/16 matrix. Self-emulsifying systems are conventionally prepared using the melt-mixing process using a paddle-mixer. Extrusion process offers a better mixing efficiency as compared to a paddle mixer, thus rendering a homogenous matrix with absence of drug hot spots. This yields a formulation with uniformly dispersed drug and improved and consistent performance. 40% drug loaded granules were prepared using both the processes and the release studies showed that the granules formulated using the optimized extrusion process had higher concentration of the drug post release as compared to the granules prepared using the conventional process. Both the formulations showed pH dependent release profiles.

v. Comparison of Use of Physical Mixture Against Granules as Raw Feed for 3D Printing

The end goal of the processed granules was to be filled into capsules for oral delivery or the granule processing to be coupled in tandem to a 3D printing platform that could personalize drug delivery for patient-specific needs. The processed granules were compared to use of a physical blend of the same composition after 3D printing as an oral tablet under the same printing conditions. Use of physical mixture as the direct feed for printing rendered tablets with a non-uniform drug distribution, along with highly variable release profile. The increased apparent solubility observed can be attributed to the slow melt mixing due to the pressure created inside the nozzle (Henry et al., 2021). The tablets processed using physical mixture showed variations because of inefficient drug flow inside the barrel due to the low melt viscosity of the lipid. Such variations make the extrusion printing of lipids a challenging process. The formulated granules show a narrow variability in their release profile along with higher concentration of the released drug as compared to the former. The reason behind the improvements of drug distribution and release was that the granules played a vital role in improving lipid flowability along with already being a formulation with uniformly distributed drug.

vi. Cell Viability and Permeability Studies

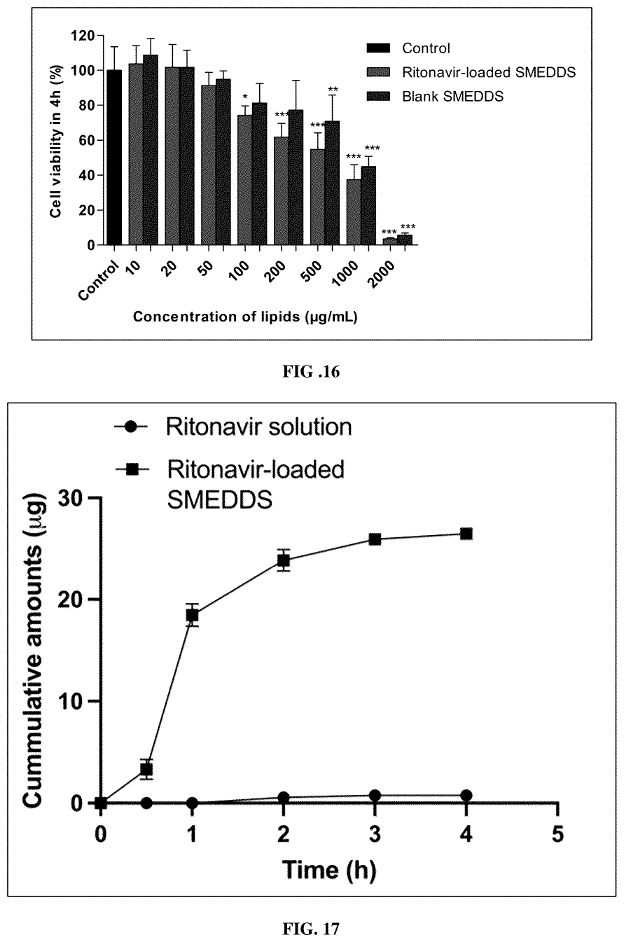

The cellular viability of the ritonavir-loaded SMEDDS and blank SMEDDS was examined in Caco-2 cells representing the enterocytes. To evaluate their possible concentration-dependent cellular toxicities, these formulations were tested at different concentrations of lipids, ranging from 10 to 2,000 μg/mL across the Caco-2 cells at an incubation time of 4 h, and the results are presented in FIG. 16. The findings reveal the concentration-dependent decrease in cell viability for both formulations. Both formulations, at lipid concentrations>200 μg/mL, exhibited significant cytotoxicity (*** p<0.001 vs. control, and ** p<0.001 vs. control). However, neither formulation induced significant cytotoxicity at lipids concentrations<100 μg/mL. As no significant cytotoxicity was observed, both formulations at a concentration of 50 μg/mL were used for further cellular studies.

The cumulative permeation profiles of the ritonavir from the SMEDDS across the Caco-2 cell monolayers are illustrated as a function of time (FIG. 17). The cumulative amount permeated by the ritonavir-loaded SMEDDS was ˜35-fold higher than that of the ritonavir solution.

TABLE 5
Permeation parameters were calculated from permeability
studies of SMEDDS across Caco-2 cell monolayers.

| Formulation | Pₐₚₚ (cm/s) × 10⁻⁶ | ER |
|---|---|---|
| Ritonavir solution | 1.89 ± 0.02 | 1.00 |
| Ritonavir-loaded SMEDDS | 65.68 ± 1.28*** | 34.75 |

As depicted in Table 5, the potential of the SMEDDS for facilitating ritonavir permeability across the Caco-2 cell monolayers over a period of 4 h, was evaluated by calculating the permeation parameters such as Pₐₚₚ and ER. The increments of Pₐₚₚ were observed to be in the order of Ritonavir-loaded SMEDDS>Ritonavir solution. The Ritonavir-loaded SMEDDS had the highest Pₐₚₚ, which was significantly greater than those of the Ritonavir solution (*** p<0.001 vs. Ritonavir solution) with an ER of ˜35-fold.

### Example 2-Selective Laser Melting (SLM) 3D-Prinitng of Dosage Form from the Lipid-Based Granues Prepared Through Hot-Melt Extrusion Process

### A. Materials and Methods

i. Materials

Acetaminophen (>98% purity) was obtained and used as a model drug from Sigma-Aldrich®, Inc. (Lot no. MKCJ5427, Sigma-Aldrich®, MO, USA). Gattefosse (Paramus, NJ, USA) kindly provided Compritol 888 ATO (batch no. 19158), a spray-dried fine white powder with melting point (M.P) 65-77° C. The potassium aluminum silicate-based pearlescent pigment Candurin® Gold Sheen was used as an energy absorbing excipient and purchased from Merck (Lot no. W150645X08, Merck KGaA, 64271 Darmstadt, Germany). For buffer preparation, sodium phosphate monobasic, sodium hydroxide, and sodium chloride were obtained from Fisher Scientific (Waltham, MA, USA), and the company also supplied methanol and acetonitrile (HPLC grade, >99.7%). All other chemicals and reagents were ACS grade or above and were used as supplied.

ii. Twin-Screw Hot-Melt Granulation Process

For the HME granulation process, four different batches of physical mixture (excluding Candurin, referred to as PM-I) were initially prepared by varying the composition of ACM and Compritol, as shown in Table 6 for the granulation process. Then the PM-I were passed through 250 μm pore size sieves to break any large agglomerates that were particularly observed with pure ACM powder. For uniform blending, the 200 g of each PM-I were blended manually for some minutes using a zip-lock bag and then gently mixed with the help of mortar and pestle. The PM-I was feed into the feeder of Leistritz ZSE 12 mm twin screw co-rotating twin-screw extruder (Leistritz Advanced Technologies Corp., Nuremberg, Germany). The feed rate was calibrated and maintained at 8 g/min (gravimetric) for all the runs and the same screw designs with an open discharge were used. Additionally, the temperature profile, and the screw speed (ranging from 25 to 100 rpm) were changed to understand their effects on granules size distribution. The HME-based granulation process was operated using a modified screw design with kneading zones thought the barrel to induce high shear mediated mixing, melting, and breakage of the formed granules. A series of preliminary runs were conducted by altering the temperature and screw speed, to find a suitable range for the process optimization process. The granules were examined, and once the process was established (torque<8%), the granules were collected in a pan and stored in a zip-lock bag. The physical mixture or physical blend and the collected granules were subjected to bulk property testing, sizing, and physicochemical characterization.

TABLE 6
Compositions of the different formulations at varying lipid
compositions keeping Candurin ® constant

| Compritol 888 ATO | Acetaminophen | Candurin ® | |
|---|---|---|---|
| (glyceryl dibehenate) | (% w/w) | (% w/w) | Formulations |
| 45 | 53.5 | 1.5 | FA1 |
| 35 | 63.5 | 1.5 | FA2 |
| 25 | 73.5 | 1.5 | FA3 |
| 15 | 83.5 | 1.5 | FA4 |

### B. Micromeritic Properties Testing

i. Particle Size Distribution (PSD) of Granules

An ASTM-approved set of sieves (#12-#100) and an Advantech L3P sonic sifter separator (Advantech Manufacturing Inc.; New Berlin, WI, U.S.) were used to sieve the granules for 5 mins at 4 amplitudes. The weight of the granules retained on the sieves was measured to estimate the % mass fraction of granules in the batch prior to milling.

ii. Study of Micromeritic Properties of Prepared Granules

To measure the bulk density of the powder, the powder was filled into a 100 mL graduated cylinder, slanting the cylinder to avoid the influence of gravity. The initial volume occupied by the powder was measured, which represents the bulk volume of the powder and the mass of the sample was recorded. The powder sample was then subjected to tapping using a tapping apparatus equipped with a digital counter (Varian Tap Density Tester 50-1000) for 250, 500 and 1000 taps, where no further changes was observed (ASTM) protocol (D7481-18). The final volume after tapping the powder was recorded. All of these experiments were conducted three times (n=3). Equations (1) and (2) gives the bulk and tapped density. Similarly, Hausner's ratio and Carr's index were also determined using equations (3) and (4).

$$Bulk density ( ρ b ) = \frac{weight ( g )}{bulk volume ( Vb , {cm}^{3} )} \qquad ( 1 )$$

$$Tapped density ( ρ t ) = \frac{weight ( g )}{tapped volume ( Vt , {cm}^{3} )} \qquad ( 2 )$$

$$Hausner ' s ratio ( HR ) = \frac{tapped density ( ρ t )}{bulk density ( ρ b )} \qquad ( 3 )$$

$$Compressibility index ( CI ) = ( 1 - \frac{bulk density}{apped density} ) \times 100 \% \qquad ( 4 )$$

### C. Selective Laser Melting (SLM) 3D Printing

The granules were sieved through (#125 μm and #65 μm) and only granules lying between 125-65 μm were used for 3D printing. The granules containing drug (ACM), and lipid (Compritol 888 ATO), were mixed with a fixed amount of Candurin® (1.5% w/w) and then blended for 15 mins to ensure uniform mixing. This blend is referred herein as physical mixture II (PM-II). Roughly 180 g of PM-II was then introduced to the feed compartment (110× 110× 110 mm) of the desktop SLS 3D printer, (Sintratec kit, Sintratec, Switzerland) comprising of a 2.3 W 455 nm laser (λ=445 nm).

Tinkercad, a freely available online software by Autodesk Inc. (San Francisco, CA, USA), was used to create flat-faced cylindrical tablets measuring 12 mm in diameter and 6 mm in height. After exporting the templates as stereolithography (.stl) files, they were exported onto the desktop computer connected to the SLS 3D printer and copied to print 12 tablets in one batch at different regions on the print chamber without overlapping. Moving forward, the printing conditions like, the hatching spacing, hatching offset, number of perimeters and perimeter offset were all set at 50 μm, 120 μm, 1, and 200 μm, respectively. Following the initial screening, the printing conditions were adjusted to laser speed=50 mm/s, layer thickness of 150 μm, chamber temperature and print surface temperature of 50° C. and 65° C., respectively, which was below the melting point of the lipid and the drug. In our prior studies and reported by others, it has been observed that the chamber temperature if kept close or above the glass transition point (T_{g}) of the polymer (herein Mp of lipid) or Mp of a drug, results in the formation of agglomerates due to the softening of the polymer or partial or complete melting of the lipid/drug, thereby, preventing the printing process. These process setting were kept constant for each printing cycle and the left-over powder was collected and mixed with a virgin powder (70:30 ratio) to print another batch after each completion cycle. During each printing cycle, a roller spreads the powder from the feed compartment to the print compartment that creates an even flat surface of powder, following which the laser activated from the top and melts the particles at a predefined printing condition, printing the first layer. Subsequently, the print compartment lowers down, while feed compartment rises up and roller moves back and forth from the feed compartment to the print compartment to spread another powder layer over the prior printed surface. The laser gets activated again to print the second layer on top of the first layer to fuse these layers together. Layer-by-layer, this process is continued until all tablets were printed in accordance with the CAD model.

In addition, a Dino-Lite™ optical microscope (AnMo Electronics Corporation, New Taipei, Taiwan) with Dino Capture Software was used to take the images of the drug particles. lipid, granules, and the printed tablets subjected on a black background.

### D. Morphology and Porosity Evaluation

i. Polarized Light Microscopy (PLM)

The morphology and crystallinity of ACM, lipid, and granules was investigated utilizing the PLM comprising of Olympus BX53 photomicroscope (Olympus America Inc., Webster, TX, U.S.) equipped with Bertrand lens (530 nm compensator), a 10× objective lens, and a 20× magnification lens. The powdered samples were evenly distributed as a single layer onto a transparent glass slide and the excess powder was removed. The glass slide was then clamped onto the stage holder and examined with a 10× magnification lens. After adjusting the light and scanning the surface, a suitable zone was selected to capture the images (Linksys 32 software®) with a 10× magnification as well as a zoomed images with 20× magnification.

ii. Scanning Electron Microscopy (SEM)

The surface morphology of the powdered materials (drug, lipid, and granules) was assessed using an SEM (Quanta FEG 650 ESEM, FEI Company, Hillsboro, USA). A few grams of samples were placed on an aluminum stub with double-sided adhesive carbon tape and then, gold-sputtered under vacuum for 45s at 40 mA (EMS, Hatfield, PA, U.S.). The samples were transferred to the SEM sample stage, the instrument was purged, and the settings were adjusted: 10 mm working distance, 2-5 kV voltage, and 3.5-4.5 mA emission current. The images were taken at various magnifications (100×-500×) from different regions of the samples.

Furthermore, a Dino-Lite™ optical microscope (AnMo Electronics Corporation, New Taipei, Taiwan) with Dino Capture Software was used to capture the images of the drug particles, lipid, granules, and the printed tablets subjected on a black background.

iii. Micro-CT Imaging

The closed and open porosity, and internal pore structure of the 3D-printed tablets before and after 12 h dissolution has been examined using a ZEISS Xradia 620 Varsa (ZEISS Microscopy; Jena, Germany) at The University of Texas at Austin. With a voxel size of 6.7 microns, each tablet dataset had 1,229 total slices. The following settings were used for the sample throughout the scanning procedure. Flat panel, 70 kV, 8.5W, 0.04s acquisition time, 5 samples/view, detector 174.185 mm, source −17.063 mm, XYZ [73, −41435, 363], camera bin 1, angle±180, 3001 views, LE1 filter, dithering, and multi-reference. Reconstructed with center shift 0.318, beam hardening 0, theta 0, byte scaling [−0.01, 0.1], binning 1, and recon filter smooth (kernel size=0.7) were used to reconstruct the image. The images were captured in 16- and 8-bit. TIFF file but only 16-bit images were used for porosity analysis using Dragonfly software (Comet Technologies, Quebec, Canada), which included 3D reconstruction and digital segmentation.

### E. Drug Content and In-Vitro Drug Release Studies

In brief, a few tablets were cursed in a mortar and pestle before being transferred a known mass of the tablet powder into 100 mL volumetric flasks containing the mobile phase. The samples were sonicated for a few minutes before being adjusted to the marked 100 mL (n=3). 1 mL of aliqotes were withdrawn and filtered using a 0.22 μm filters. The drug content was then quantified using a reverse-phase HPLC system (ThermoFisher Vanquish HPLC system, Thermo Fisher, Waltham, MA, USA) comprising of a L×I.D. 250×4.6 mm, 5 μm particle size C-18 column (Discovery® C-18, Supelco™, Millipore Sigma, MA, USA) kept at 35° C. The mobile phase is a 70:30 (v/v) mixture of methanol and deionized water with 0.1% orthophosphoric acid (pH 2.3), respectively, eluted at 0.8 mL/min (isocratic) for 9 mins. The injection volume was kept same at 20 μL and the absorbance signals were detected with a UV-Vis spectrophotometer detector (ThermoFisher Vanquish HPLC system, Waltham, MA, USA) at λ=247 nm. As in our prior study, a six-point standard calibration curve was developed that was found linear (R²=0.999). The drug loading was calculated.

The in vitro drug release studies of the 3D printed tablets was carried out using USP type-II apparatus. In brief, the tablets (n=5) were weighed before being dropped into the 1L dissolution vessels (Varian VK 7000 dissolution system, Varian, Inc., Cary, NC, U.S.) containing 750 mL of HCl-KCl buffer (pH 2, 0.1M HCl-KCl buffer) for 2h kept at 37±5C, and agitated at 50 rpm. After that, 150 mL of phosphate buffer (pH 6.8, 0.1M) was added to each vessel to raise the pH from 2 to 6.8, for a total volume of 900 mL. It is essential to note that the phosphate buffer should be made for a final volume of 900 mL, so the above-mentioned 150 mL volume is the concentrated buffer required for shifting the pH. At specified time intervals (5, 15, 30, 60, 120, 240, 720, and 1440 mins), 1.5 mL samples were collected using an autosampler (Vankel VK 8000, Agilent Technologies, USA), and an equivalent volume of fresh medium was added to the vessels. Following, the samples were filtered through 0.22 m PTFE syringe filters and diluted 2× with the mobile phase before subjecting to the RP-HPLC for drug quantification.

### F. Solid-State Characterization

i. Differential Scanning Calorimetry (DSC)

The samples (drug, lipid, drug-loaded granules, PM-II, and crushed tablet powder), weighing roughly 3-4 mg, were kept into T-zero aluminum pans (DSC consumables Inc., MS, USA), weighed (Sartorius 3.6P microbalance, Göttingen, Germany) and properly sealed with the lids. The same set of empty aluminium pan was used as a reference. Both the pans were transferred to a DSC Q20 equipment (TAR instruments, New Castle, DE, U.S.). The samples underwent constant ramp heating at 5° C./min between 20 and 200° C. temperature range under continuous a nitrogen flow at 50 mL/min. The acquired data were analyzed using Universal Analysis 2000 software for TA® devices, and figures were plotted as temperature (C) versus heat flow (W/g).

ii. Powder X-Ray Diffraction (PXRD)

A benchtop Rigaku MiniFlex 600 II (Rigaku Corporation, Tokyo, Japan) fitted with a Cu Kα X-ray source (λ=1.5418 A) was used to gather the X-ray diffraction patterns of the samples. The powder samples were distributed over the sample cells, gently flattened to prepare an even disk surface using a glass slide and then transferred to the benchtop XRD instrument sample holders. The XRD settings were as follows: scan range between 5 and 55° (2θ degree), scan speed of 2°/min, step size of 0.02, voltage and current were maintained at 45 kV and 15 mA, respectively.

iii. Fourier Transform Infrared Radiation (FT-IR)

FT-IR was utilized to investigate peak shifts caused by thermal processes during HME granulation and/or SLM 3D printing. In brief, an Infinity Gold FTIR Spectrometer (Thermo Mattson) equipped with Narrow Band MCT detector was used to investigate for intermolecular interaction, and degradation of the powdered samples. Each sample (roughly 15-20 mg) was dispersed onto the cell after cleaning with isopropanol to avoid contamination from earlier samples. After correcting for background, a total of 64 scans with a resolution of 4 cm⁻¹ were collected between 700 and 4000 cm⁻¹ wavenumbers and analyzed using WINFIRST software.

### G. Results

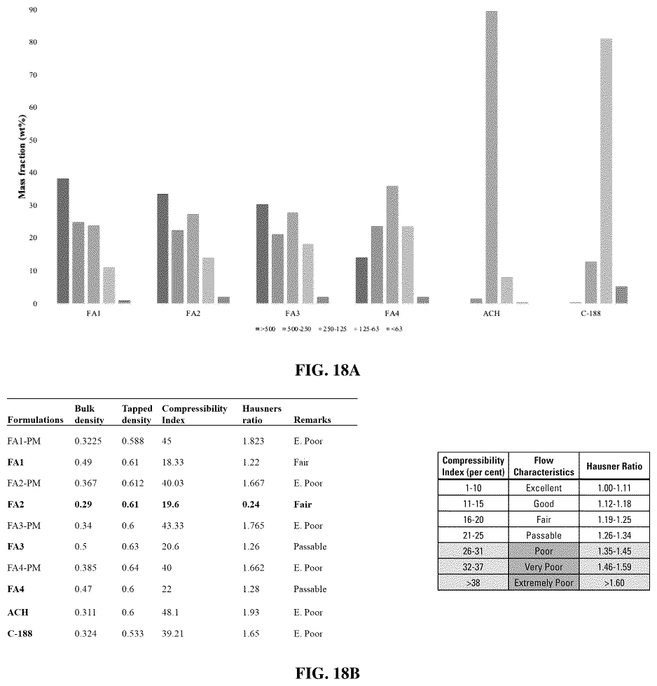

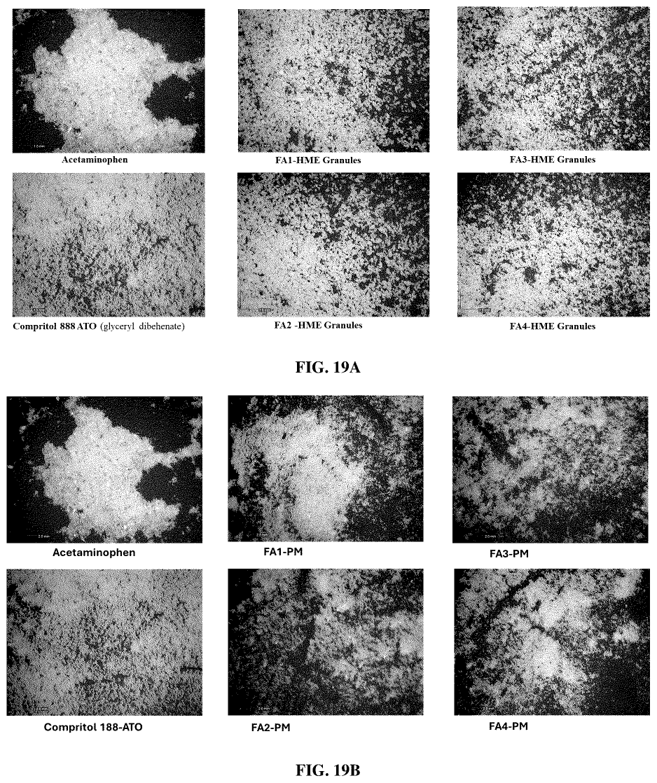

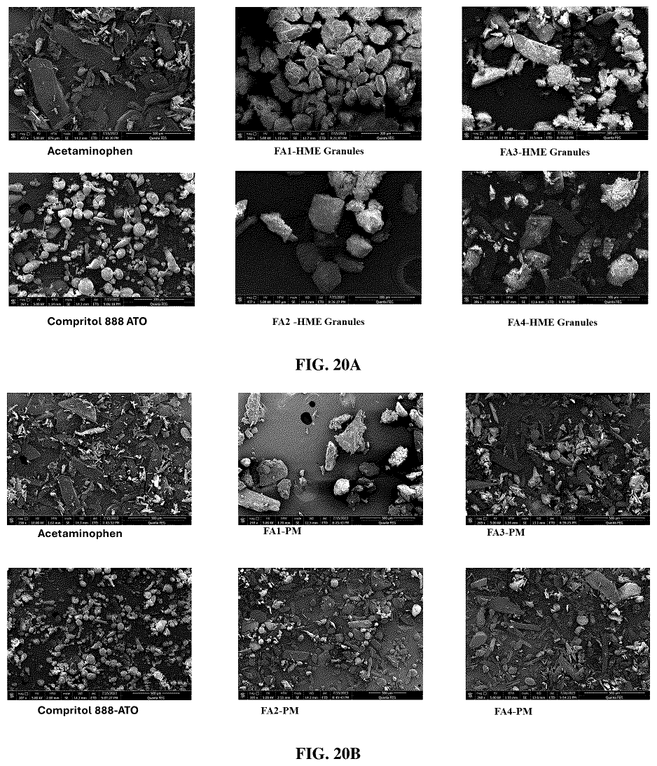

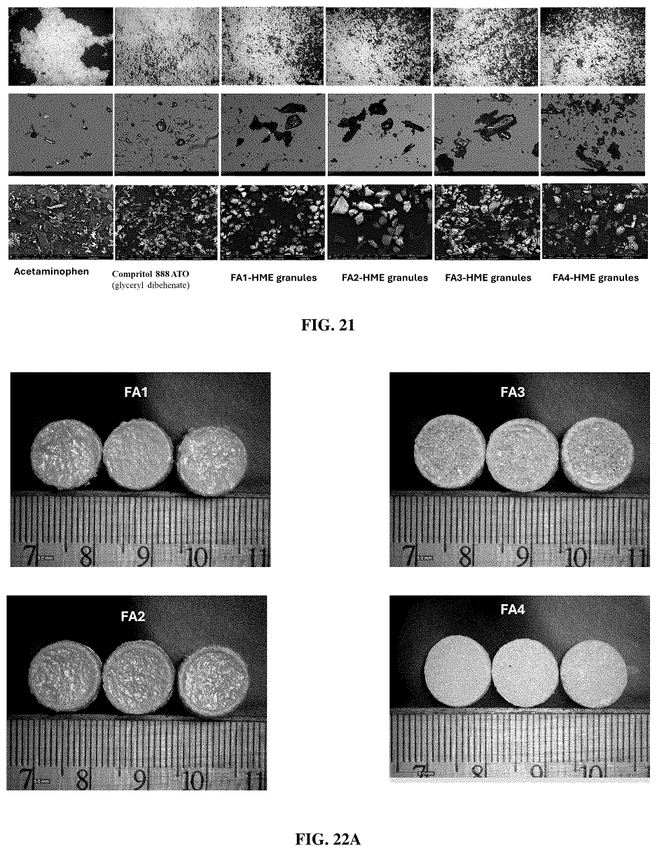

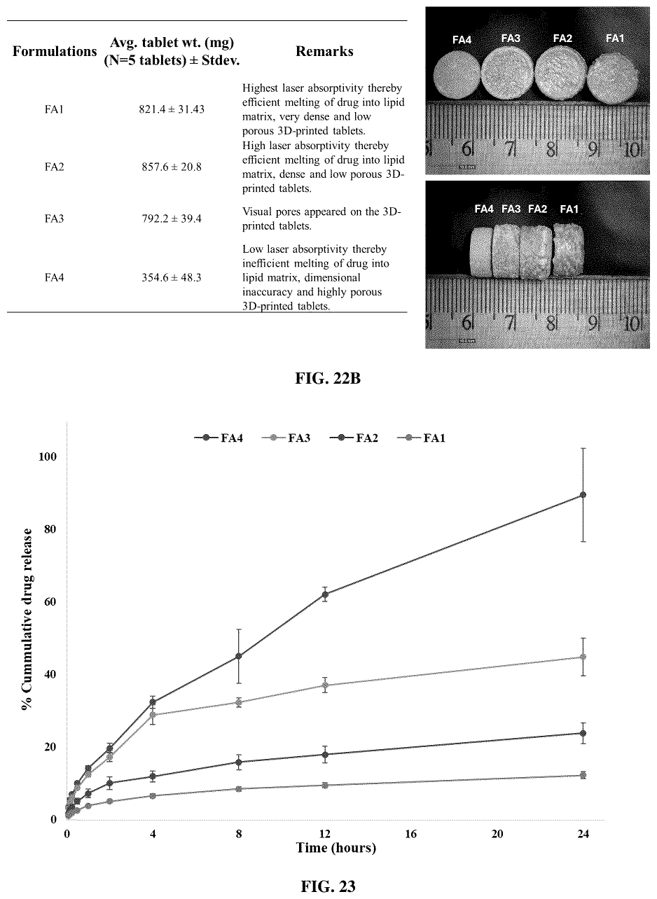

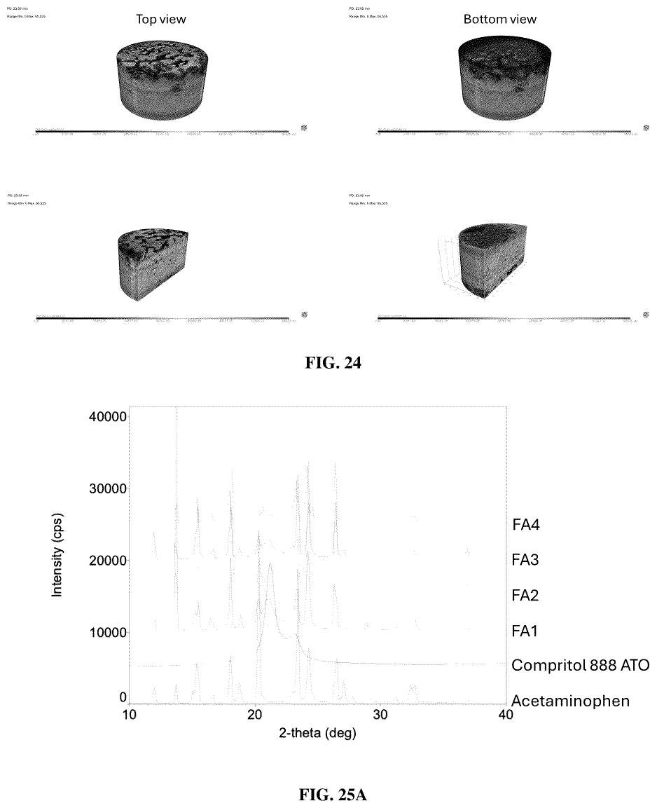

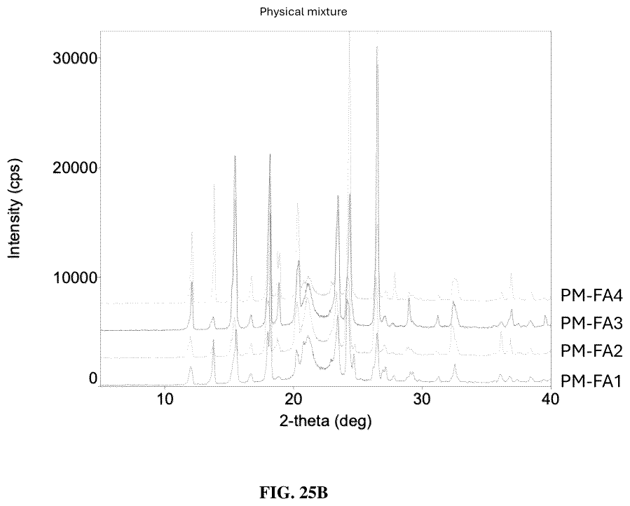

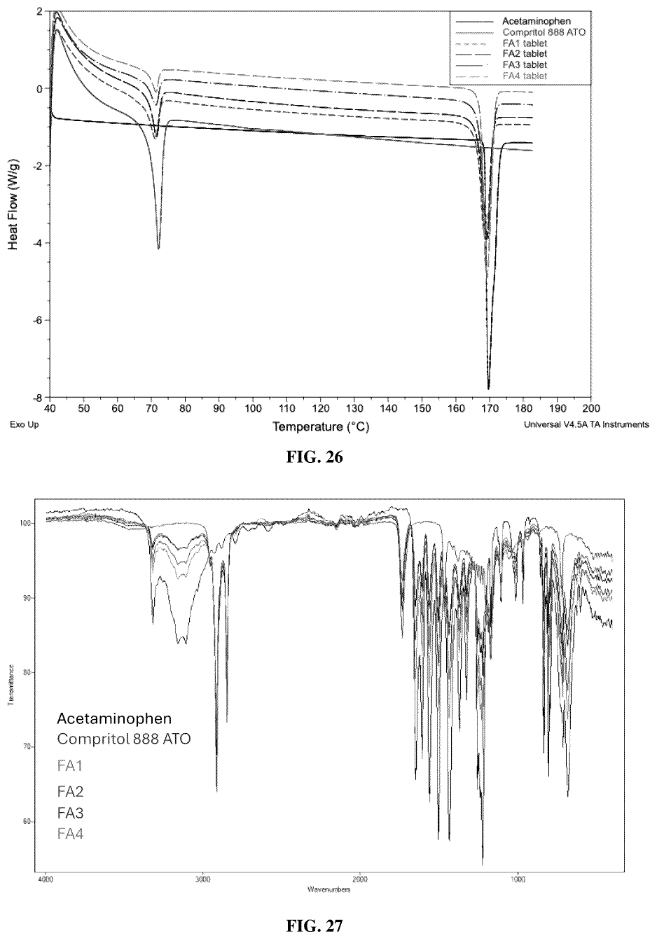

FIG. 1 shows the process of preparing powder-based 3D printing compositions using acetaminophen, as a model drug. The hot-melt extrusion process and 3D printing processes are highlighted in FIG. 1. The effects on lipid levels in the processing of granulation are shown in FIGS. 18A and 18B. These results shows that extremely poor flowing characteristics of drug and lipid are improved by granulation process. The morphology of the prepared granules compositions before i.e., physical mixture and after hot-melt extrusion processing are shown in FIGS. 19-21-23 through dino-lite microscopy, polarized light microscopy (PLM), and scanning electron microscopy (SEM) images. The morphology of the 3D-printed tablets from the processed compositions (Table 6), their average weight, and visual observations are then shown in FIGS. 22A and 22B. The dissolution profile of the printed tablets from different compostions are shown in FIG. 23. FIG. 24 shows the micro-CT images of the 3D printed tablet (FA2 formulation) post 24 h of dissolution studies. Many cracks can be observed particularly on the top and bottom portion of the tablet which indicates that there was penetration of dissolution fluid from these regions that causes the release of drug into the media over an extended period of time (beyond 24 h). Such as extended release with low burst release effect is difficult to achieve in pharmaceutical 3D printed products, particularly with powder-based additive manufacturing techniques due to their highly porous nature (Giri et al. 2022). The XRD graph shows that both the granules and the tablets post 3D printing are in crystalline state and there is no significant changes in their solid-state properties as shown in FIGS. 25A and 25B. This is further supported by DSC thermograms in FIG. 26. Finally, the FTIR spectra in FIG. 27 shows the retention of the major characteristic acetaminophen peaks as well as the lipid (compritol 888 ATO) peaks in all of the 3D-printed tablet formulations, which indicates absence of chemical reactions or undesired degradation between the drug and carriers post printing.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

### REFERENCE

U.S. Pat. No. 4,009,139
U.S. Pat. No. 5,571,334
U.S. Pat. No. 8,486,423
U.S. Pat. No. 9,339,440
PCT Patent Application No. WO 2020/122950
Silva et al., Preparation of a solid self-microemulsifying drug delivery system by hot-melt extrusion. Int J Pharm.; 541 (1-2): 1-10, 2018.
Shah et al., A theoretical basis for a biopharmaceutic drug classification: The correlation of in vitro drug product dissolution and in vivo bioavailability, Pharm Res 12, 413-420, 1995-Backstory of BCS. AAPS Journal. 2014; 16 (5): 894-898, 2014.
Markovic et al., Bcs class iv oral drugs and absorption windows: Regional-dependent intestinal permeability of furosemide. Pharmaceutics.; 12 (12): 1-16, 2020.
Shadambikar et al., Formulation development of itraconazole PEGylated nano-lipid carriers for pulmonary aspergillosis using hot-melt extrusion technology. Int J Pharm X.: 3, 2021.
Nyavanandi et al., Impact of hydrophilic binders on stability of lipid-based sustained release matrices of quetiapine fumarate by the continuous twin screw melt granulation technique. Advanced Powder Technology.: 32 (7): 2591-2604, 2021.
Araújo et al., Effects of Formulation and Manufacturing Process on Drug Release from Solid Self-emulsifying Drug Delivery Systems Prepared by High Shear Mixing. AAPS PharmSciTech.: 22 (8), 2021.
Sarabu et al., A One-Step Twin-Screw Melt Granulation with Gelucire 48/16 and Surface Adsorbent to Improve the Solubility of Poorly Soluble Drugs: Effect of Formulation Variables on Dissolution and Stability. AAPS PharmSciTech.: 22 (3), 2021.
Maji et al., Solid self emulsifying drug delivery system: Superior mode for oral delivery of hydrophobic cargos. Journal of Controlled Release. 2021:337:646-660, 2021.
Muhindo et al., Continuous production of raloxifene hydrochloride loaded nanostructured lipid carriers using hot-melt extrusion technology. J Drug Deliv Sci Technol.; 65, 2021.
Aloisio et al., Development of self-microemulsifying lipid-based formulations of trans-resveratrol by systematically constructing lipid-surfactant-water phase diagrams using long-chain lipids. Drug Dev Ind Pharm.: 47 (6): 897-907, 2021. doi: 10.1080/03639045.2021.1934866
Meirinho et al., Self-Emulsifying Drug Delivery Systems: An Alternative Approach to Improve Brain Bioavailability of Poorly Water-Soluble Drugs through Intranasal Administration. Pharmaceutics.: 14 (7), 2022.
Jörgensen et al., Cosolvents in Self-Emulsifying Drug Delivery Systems (SEDDS): Do They Really Solve Our Solubility Problems? Mol Pharm.; 17 (9): 3236-3245, 2020.
Nealey et al., Trade secrets in life science and pharmaceutical companies. Cold Spring Harb Perspect Med.; 5 (4), 2015.
Park et al., Current status of supersaturable self-emulsifying drug delivery systems. Pharmaceutics.: 12 (4), 2020.
Alskär et al., Tools for Early Prediction of Drug Loading in Lipid-Based Formulations. Mol Pharm.; 13 (1): 251-261, 2016. IJPhS-77-249.
Ameta et al., Recent Advances in Improving the Bioavailability of Hydrophobic/Lipophilic Drugs and Their Delivery via Self-Emulsifying Formulations. Colloids and Interfaces.: 7 (1), 2023.
Panigrahi et al., Gelucire: A versatile polymer for modified release drug delivery system. Futur J Pharm Sci.: 4 (1): 102-108, 2018.
Nyavanandi et al., Twin Screw Melt Granulation: A Single Step Approach for Developing Self-Emulsifying Drug Delivery System for Lipophilic Drugs. Pharmaceutics.; 15 (9): 2267, 2023.
Wang et al., Study on the relationships between the oil HLB value and emulsion stabilization. RSC Adv.: 13 (35): 24692-24698, 2023.
Kale and Patravale, Design and evaluation of self-emulsifying drug delivery systems (SEDDS) of nimodipine. AAPS PharmSciTech.; 9 (1): 191-196, 2008.
Upadhyay et al., Gelucire: An Alternative Formulation Technological Tool for Both Sustained and Fast Release of Drugs in Treating Diabetes Mellitus Type II Disease. Vol 72.; 2013.
Chandwani and Shuter, Lopinavir/Ritonavir in the Treatment of HIV-1 Infection: A Review. Vol 4.; 2008.
Law et al., Physicochemical Considerations s in the Preparation of Amorphous Ritonavir+Poly(Ethylene Glycol) 8000 Solid Dispersions, J. Pharm Sci: 90 (8): 1015-25, 2001.
Maniruzzaman et al., A Review of Hot-Melt Extrusion: Process Technology to Pharmaceutical Products. ISRN Pharm.: 2012:1-9, 2012.
Sarabu et al., An update on the contribution of hot-melt extrusion technology to novel drug delivery in the twenty-first century: part II. Expert Opin Drug Deliv.; 16 (6): 567-582, 2019.
Farinha et al., Production of Lysozyme-PLGA-Loaded Microparticles for Controlled Release Using Hot-Melt Extrusion. AAPS PharmSciTech.; 21 (7), 2020.
Chauhan et al., The preparation of lipid-based drug delivery system using melt extrusion. Drug Discov Today.: 25 (11): 1930-1943, 2020.
Rosch et al., Development of an immediate release excipient composition for 3D printing via direct powder extrusion in a hospital. Int J Pharm.; 643, 2023.
Pistone et al., Direct cyclodextrin based powder extrusion 3D printing of budesonide loaded mini-tablets for the treatment of eosinophilic colitis in paediatric patients. Int J Pharm.; 632, 2023.
Henry et al., Extrusion-based 3D printing of oral solid dosage forms: Material requirements and equipment dependencies. Int J Pharm.; 598, 2021.
Wang et al., Preparation of core-shell controlled release tablets using direct powder extrusion 3D printing techniques. J Drug Deliv Sci Technol.; 88, 2023.
Bhujbal et al., Pharmaceutical amorphous solid dispersion: A review of manufacturing strategies. Acta Pharm Sin B.; 11 (8): 2505-2536, 2021.
McDonagh et al., Direct Granule Feeding of Thermal Droplet Deposition 3D Printing of Porous Pharmaceutical Solid Dosage Forms Free of Plasticisers. Pharm Res.; 39 (3): 599-610, 2022.
Prajapati et al., A comparative evaluation of mono-, di- and triglyceride of medium chain fatty acids by lipid/surfactant/water phase diagram, solubility determination and dispersion testing for application in pharmaceutical dosage form development. Pharm Res.; 29 (1): 285-305, 2012.
Kallakunta et al., An update on the contribution of hot-melt extrusion technology to novel drug delivery in the twenty-first century: part I. Expert Opin Drug Deliv.; 16 (5): 539-550, 2019.
Lipid-Based Formulations A Winning Strategy to Overcome Oral Bioavailability Challenges People Make Our Name.
Repka et al., Melt extrusion with poorly soluble drugs—An integrated review. Int J Pharm.; 535 (1-2): 68-85, 2018.
Suk et al., PEGylation as a strategy for improving nanoparticle-based drug and gene delivery. Adv Drug Deliv Rev.; 99:28-51, 2016.
Crowley et al., 2007.
DiNunzio et al., 2010a.
DiNunzio et al., 2010b.
DiNunzio et al., 2010c.
DiNunzio et al., 2010d.
Gibson et al., 2015.
Fina et al., 2018.
Giri et al. 2022.
Hughey et al., 2010.
Hughey et al., 2011.
Keen et al., 2013.
LaFountaine et al., 2016a.
LaFountaine et al., 2016b.
Lang et al., 2014.
Prasad et al., 2016.
Repka et al., 2007.
Vynckier et al., 2014.
Williams et al., 2015,
Yap et al., 2015.

1. A method of preparing a pharmaceutical composition comprising:
(A) obtaining a precursor composition comprising:
(1) one or more active pharmaceutical ingredient;
(2) one or more lipid components; and
(3) one or more energy absorbing material;
(B) extruding the precursor composition in an extruder to obtain a feedstock composition; and
(C) subjecting the feedstock composition to a sintering process in an additive manufacturing to obtain the pharmaceutical composition.

2. The method of claim 1 further comprising subjecting the precursor solution to a sieve before extrusion.

3. The method of claim 1, wherein the pharmaceutical composition comprises one lipid.

4. The method of claim 1, wherein the pharmaceutical composition comprises two or more lipids.

5. The method according to claim 1, wherein the energy absorbing excipient is an inorganic material.

6. The method according to claim 1, wherein the precursor composition comprises an amount of the active pharmaceutical ingredient from about 1% to about 98%.

7. The method according to claim 1, wherein the precursor composition comprises an amount of the one or more lipids from about 1% to about 70%.

8. The method according to claim 1, wherein the precursor composition comprises an amount of the energy absorbing excipient from about 0.1% to about 10% w/w.

9. The method according to claim 1, wherein the melting process is a selective laser melting process.

10. The method of claim 9, wherein the melting or sintering process comprises using a laser to heat the feedstock composition.

11. The method according to claim 1, wherein the sintering process comprises using a laser with a hatch spacing from about 5 μm to about 150 μm, a hatching offset from about 5 μm to about 500 μm, a number of perimeters from about 0 to about 10, a perimeter offset from about 10 μm to about 750 μm, applying a layer of feedstock composition with a layer thickness from about 10 μm to about 750 μm, and/or passing a laser over the feedstock composition with a laser speed from about 5 mm/s to about 150 mm/s.

12. The method according to claim 1, wherein the melting or sintering process comprises a chamber with a chamber temperature that is below the melting point or glass transition temperature of the feedstock composition.

13. The method according to claim 1, wherein the melting process comprises a chamber with a layer of the feedstock composition applied to a print surface.

14. The method according to claim 1, wherein the melting process comprises applying a layer of the feedstock composition to a surface in a chamber.

15. The method according to claim 1, wherein the melting process comprises applying a second layer of the feedstock composition after a laser has been passed over the first layer of the feedstock composition.

16. The method of claim 15, wherein melting process comprises repeating the applying of another layer of the feedstock composition after the laser has been passed over the first layer of the feedstock composition multiple times.

17. A method of preparing a pharmaceutical composition comprising:
(A) admixing a lipid and a BCS Class II or BCS Class IV drug at an elevated temperature to form a precursor solution; and
(B) extruding the pharmaceutical composition to obtain one or more granules of the pharmaceutical composition.

18. The method of claim 17, wherein the method further comprises subjecting the pharmaceutical composition to an additive manufacturing process.

19. The method of claim 18, wherein the additive manufacturing process comprises a printing speed from about 1 mm/s to about 20 mm/s and/or the additive manufacturing process comprises a print pressure from about 10 kPa to about 1 MPa.

20. A method of treating a disease or disorder comprising administering to the patient a therapeutically effective amount of a composition prepared according to the method of claim 1.
